(19) United States
(12) Patent Application Publication
(10) Pub. No.: US 2006/0160790 A1
(43) Pub. Date: Jul. 20, 2006

(54) Thioureas as factor Xa inhibitors

(75) Inventors: Yonghong Song, Foster City, CA (US); Bing-Yan Zhu, Palo Alto, CA (US); Chhaya Bhakta, Hayward, CA (US); Robert M. Scarborough, Half Moon Bay, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Portola Pharmaceuticals, Inc., South San Francisco, CA (US)

(21) Appl. No.: 11/298,296

(22) Filed: Dec. 7, 2005

Related U.S. Application Data
(60) Provisional application No. 60/634,150, filed on Dec. 7, 2004.

(51) Int. Cl. A61K 31/55; A61K 31/4709; A61K 31/454; A61K 31/4015; C07D 403/02; C07D 401/02

(52) U.S. Cl. 514/212.08; 514/312; 514/424; 540/524; 546/153; 546/207; 548/547; 514/326

(57) ABSTRACT

The present invention is directed to compounds represented by Formula I and pharmaceutically acceptable salts, solvates, hydrates, and prodrugs thereof which are inhibitors of Factor Xa. The present invention is also directed to and intermediates used in making such compounds, pharmaceutical compositions containing such compounds, methods to prevent or treat a number of conditions characterized by undesired thrombosis and methods of inhibiting the coagulation of a blood sample.

### CROSS-REFERENCES TO RELATED APPLICATIONS

This application claims the benefit of U.S. Patent Application No. 60/634,150, filed Dec. 7, 2004, the content of which is incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Hemostasis, the control of bleeding, occurs by surgical means, or by the physiological properties of vasoconstriction and coagulation. This invention is particularly concerned with blood coagulation and ways in which it assists in maintaining the integrity of mammalian circulation after injury, inflammation, disease, congenital defect, dysfunction or other disruption. Although platelets and blood coagulation are both involved in thrombus formation, certain components of the coagulation cascade are primarily responsible for the amplification or acceleration of the processes involved in platelet aggregation and fibrin deposition.

Thrombin is a key enzyme in the coagulation cascade as well as in hemostasis. Thrombin plays a central role in thrombosis through its ability to catalyze the conversion of fibrinogen into fibrin and through its potent platelet activation activity. Direct or indirect inhibition of thrombin activity has been the focus of a variety of recent anticoagulant strategies as reviewed by Claeson, G., “Synthetic Peptides and Peptidomimetics as Substrates and Inhibitors of Thrombin and Other Proteases in the Blood Coagulation System”, Blood Coag. Fibrinol., 5:411-436 (1994). Several classes of anticoagulants currently used in the clinic directly or indirectly affect thrombin (i.e. heparins, low-molecular weight heparins, heparin-like compounds and coumarins).

A prothrombinase complex, including Factor Xa (a serine protease, the activated form of its Factor X precursor and a member of the calcium ion binding, gamma carboxyglutamyl (Gla)-containing, vitamin K dependent, blood coagulation glycoprotein family), converts the zymogen prothrombin into the active procoagulant thrombin. Unlike thrombin, which acts on a variety of protein substrates as well as at a specific receptor, factor Xa appears to have a single physiologic substrate, namely prothrombin. Since one molecule of factor Xa may be able to generate up to 138 molecules of thrombin (Elodi et al., Thromb. Res. 15:617-619 (1979)), direct inhibition of factor Xa as a way of indirectly inhibiting the formation of thrombin may be an efficient anticoagulant strategy. Therefore, it has been suggested that compounds which selectively inhibit factor Xa may be useful as in vitro diagnostic agents, or for therapeutic administration in certain thrombotic disorders, see e.g., WO 94/13693.

Polypeptides derived from hematophagous organisms have been reported which are highly potent and specific inhibitors of factor Xa. U.S. Pat. No. 4,588,587 describes anticoagulant activity in the saliva of the Mexican leech, Haementeria officinalis. A principal component of this saliva was shown to be the polypeptide factor Xa inhibitor, antistasin (ATS), by Nutt, E. et al., “The Amino Acid Sequence of Antistasin, a Potent Inhibitor of Factor Xa Reveals a Repeated Internal Structure”, J. Biol. Chem., 263:10162-10167 (1988). Another potent and highly specific inhibitor of Factor Xa, called tick anticoagulant peptide (TAP), has been isolated from the whole body extract of the soft tick Ornithidoros moubata, as reported by Waxman, L., et al., “Tick Anticoagulant Peptide (TAP) is a Novel Inhibitor of Blood Coagulation Factor Xa”, Science, 248:593-596 (1990).

Factor Xa inhibitory compounds which are not large polypeptide-type inhibitors have also been reported (see e.g. Tidwell, R. R. et al., “Strategies for Anticoagulation With Synthetic Protease Inhibitors. Xa Inhibitors Versus Thrombin Inhibitors”, Thromb. Res., 19:339-349 (1980); Turner, A. D. et al., “p-Amidino Esters as Irreversible Inhibitors of Factor IXa and Xa and Thrombin”, Biochemistry, 25:4929-4935 (1986); Hitomi, Y. et al., “Inhibitory Effect of New Synthetic Protease Inhibitor (FUT-175) on the Coagulation System”, Haemostasis, 15:164-168 (1985); Sturzebecher, J. et al., “Synthetic Inhibitors of Bovine Factor Xa and Thrombin. Comparison of Their Anticoagulant Efficiency”, Thromb. Res., 54:245-252 (1989); Kam, C. M. et al., “Mechanism Based Isocoumarin Inhibitors for Trypsin and Blood Coagulation Serine Proteases: New Anticoagulants”, Biochemistry, 27:2547-2557 (1988); Hauptmann, J. et al., “Comparison of the Anticoagulant and Antithrombotic Effects of Synthetic Thrombin and Factor Xa Inhibitors”, Thromb. Haemost., 63:220-223 (1990)).

Others have reported Factor Xa inhibitors which are small molecule organic compounds, such as nitrogen containing heterocyclic compounds which have amidino substituent groups, wherein two functional groups of the compounds can bind to Factor Xa at two of its active sites. For example, WO 98/28269 describes pyrazole compounds having a terminal C(═NH)—NH₂ group; WO 97/21437 describes benzimidazole compounds substituted by a basic radical which are connected to a naphthyl group via a straight or branched chain alkylene, C(O) or SO₂ bridging group; WO 99/10316 describes compounds having a 4-phenyl-N-alkylamidino-piperidine and 4-phenoxy-N-alkylamidino-piperidine group connected to a 3-amidinophenyl group via a carboxamidealkyleneamino bridge; and EP 798295 describes compounds having a 4-phenoxy-N-alkylamidino-piperidine group connected to an amidinonaphthyl group via a substituted or unsubstituted sulfonamide or carboxamide bridging group.

There exists a need for effective therapeutic agents for the regulation of hemostasis, and for the prevention and treatment of thrombus formation and other pathological processes in the vasculature induced by thrombin such as restenosis and inflammation. In particular, there continues to be a need for compounds which selectively inhibit factor Xa or its precursors. Compounds that have different combinations of bridging groups and functional groups than compounds previously discovered are needed, particularly compounds which selectively or preferentially bind to Factor Xa. Compounds with a higher degree of binding to Factor Xa than to thrombin are desired, especially those compounds having good bioavailability and/or solubility.

### BRIEF SUMMARY OF THE INVENTION

The present invention provides in one aspect, compounds having the formula: 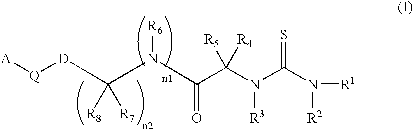
and pharmaceutically acceptable salts, hydrates, solvates and prodrugs thereof. In formula (I), the symbol R¹ represents a member selected from the group consisting of: hydrogen, —C₁₋₆alkyl, —C₀₋₆alkyl-aryl, heteroaryl and —C₂₋₆alkenyl.

The symbol R² represents a member selected from the group consisting of: a member selected from the group consisting of: —C₀₋₆alkyl-aryl, —C₃₋₈cycloalkylaryl, heteroaryl, heteroaryl-C₃₋₈cycloalkyl, —C₃₋₈cycloalkyl, —C₃₋₈cycloalkenyl, heteromonocyclyl, fused heterobicyclyl and unfused heterobicyclyl, each of which is optionally substituted with from 1 to 3 R^{2a} substituents, wherein each heteromonocyclyl, fused heterobicyclyl or unfused heterobicyclyl comprises 5 to 12 ring atoms, 1 to 4 of which are members independently selected from the group consisting of N, O and S.

The symbol R³ represents a member selected from the group consisting of: hydrogen, C₁₋₆alkyl, heteroaryl, C₂₋₆alkenyl, —C₀₋₄alkyl-C₃₋₈-cycloalkyl, —C₀₋₆alkyl-aryl, —C₀₋₆alkyl-heteroaryl, —C₀₋₆alkyl-heterocyclyl, —C₀₋₆alkyl-CO—OR^{3a}, —C₁₋₆alkyl-N(R^{3a}R^{3b}), —C₁₋₆alkyl-O—R^{3a}, —C₁₋₆alkyl-S—R^{3a}, —C₀₋₆alkyl-C(O)—N(R^{3a}R^{3b}) and —C₁₋₆alkyl-N(R^{3a})—C(O)R^{3b}.

Each R⁴ and R⁵ is a member independently selected from the group consisting of: hydrogen, —C₁₋₆alkyl, —C₂₋₆alkenyl, —C₂₋₆alkynyl, —C₀₋₄alkyl-C₃₋₈-cycloalkyl, C₁₋₆haloalkyl, —C₀₋₆alkyl-heteroaryl, —C₀₋₆alkyl-heterocyclyl, —C₀₋₆alkyl-CN, —C₀₋₆alkyl-NO₂, —C₁₋₆alkyl-O—R^{4a}, —C₁₋₆alkyl-S^{4a}, —C₁₋₆alkyl-SO₂—R^{4a}, —C₁₋₆alkyl-S(O)—R^{4a}, —C₀₋₆alkyl-CO—OR^{4a}, —C₀₋₆alkyl-C(O)—N(R^{4a}R^{4b}), —C₀₋₆alkyl-C(O)R^{4a}, —C₁₋₆alkyl-N(R^{4a}R^{4b}), —C₁₋₆alkyl-N(R^{4a})—C(O)R^{4b}, —C₁₋₆alkyl-N(R^{4a})—C(O)—N(R^{4b}R^{4c}), —C₁₋₆alkyl-N(R^{4a})—SO₂—R^{4b}, —C₁₋₆alkyl-SO₂—N(R^{4a}R^{4b}), —C₀₋₆alkyl-PO(—OR^{4a})(OR^{4b}), —C₁₋₆alkyl-N(R^{4a})—PO(OR^{4b})(—OR^{4c}), —C₀₋₆alkyl-aryl, —C₀₋₆alkyl-heteroaryl, and —C₀₋₆alkyl-heterocyclyl; or R⁴ and R⁵ can be taken together with the carbon atom to which they are attached to form a 3 to 8 membered heterocyclyl group; wherein each heterocyclyl is a 3 to 8 membered monocyclic ring or a 8-12 membered bicyclic ring, each comprising from 1 to 4 heteroatoms selected from the group consisting of O, N and S, and wherein 1 to 3 carbon or nitrogen atoms of aryl, heteroaryl and heterocyclyl are substituted with 1 to 3 R^{4d} substituents.

The letter D is a member selected from the group consisting of: a direct bond, aryl, heteroaryl, C₃₋₈cycloalkyl, C₃₋₈cycloalkylene, heteromonocyclyl, unfused heterobicyclyl, and fused heterobicyclyl; each of which is optionally substituted with 1 to 3 R⁹ substituents, wherein each heteromonocyclyl, fused heterobicyclyl or unfused heterobicyclyl comprises from 5 to 10 ring atoms, 1-4 of which are selected from the group consisting of N, O and S.

The symbol Q is selected from the group consisting of: a direct bond, —C(R^{10a}R^{10b})—, —C(O)—, —C(S)—, —C(═NR^{10a})—, —O—, —S—, —N(R^{10a})—, —N(R^{10a})CH₂—, —CH₂N(R^{10a})—, —C(O)N(R^{10a})—, —N(R^{10a})C(O)—, —SO₂—, —SO—, —SO₂N(R^{10a})—, and —N(R^{10a})—SO₂—; and at least one of D and Q is not a direct bond.

The symbol A is selected from the group consisting of: —NR^{11c}R^{11d}, —C(═NR^{11c})NR^{11a}R^{11b}, —C(═NR^{11e}R^{11f})NR^{11a}R^{11b}, —N(R^{11d})C(═NR^{11c})NR^{11a}R^{11b}, N(R^{11d})C(═NR^{11c})R^{11a}, —N(R^{11c})NR^{11a}R^{11b}, —N(R^{11c})OR^{11d}; C₁₋₆alkyl, C₂₋₆alkenyl, aryl, heteroaryl, C₃₋₈cycloalkyl, C₃₋₈cycloalkenyl, heteromonocyclyl, and fused heterobicyclyl; each of aryl, heteroaryl, heteromonocyclyl and fused heterobicyclyl, each of which is optionally substituted with 1 to 3 R^{11g}; wherein each hetercyclyl comprises from 5 to 10 ring atoms, 1-4 of which are selected from the group consisting of N, O and S.

Each R^{2a}, R^{4d}, R⁹ and R^{11g} is a member independently selected from the group consisting of: H, halo, C₁₋₆alkyl, C₂₋₆alkenyl, C₂₋₆alkynyl, C₀₋₄alkylC₃₋₈cycloalkyl, —C₁₋₄alkoxy, —O—C₀₋₂alkyl-CF₃, —C₀₋₂alkyl-CF₃, —C₀₋₂alkyl-CN, —C₀₋₂alkyl-NO₂, —C₀₋₂alkyl-NR^{12a}R^{12b}, —C₀₋₂alkyl-SO₂NR^{12a}R^{12b}, —C₀₋₂alkyl-SO₂R^{12a}, —C₀₋₂alkyl-SOR^{12a}, —C₀₋₂alkyl-CF₃, —C₀₋₂alkyl-OR^{12a}, —C₀₋₂alkyl-SR^{12a}, —O—CH₂—CH₂—OR^{12a}, —O—CH₂—CO₂R^{12a}, —N(R^{12a})—CH₂—CH₂—OR^{12b}, —C₀₋₂alkyl-C(O)NR^{12a}R^{12b}, —C₀₋₂alkyl-CO₂R^{12a}, —C₀₋₂alkyl-N(R^{12a})—C(O)R^{12b}, —C₀₋₂alkyl-N(R^{12c})—C(O)NR^{12a}R^{12b}, —C₀₋₂alkyl-C(═NR^{12c})NR^{12a}R^{12b}, —C₀₋₂alkyl-C(═NR^{12a})R^{12b}, —C₀₋₂alkyl-N(R^{12d})C(═NR^{12c})NR^{12a}R^{12b}, —C₀₋₂alkyl-N(R^{12a})—SO₂—R^{12b}, ═O, ═S, ═NR^{12a}, 5- or 6-membered aryl, 5- or 6-membered heteroaryl and 5- to 7-membered heterocyclyl, each of which is optionally substituted with a member independently selected from the group consisting of halo, CF₃, OCF₃, SCF₃, C₁₋₆alkyl, C₂₋₆alkenyl, C₂₋₆alkynyl, C₀₋₄alkylC₃₋₈cycloalkyl, C₁₋₄alkoxy, —CO₂H, —CO₂C₁₋₄alkyl, —CONR^{12a}R^{12b}, ═O, ═S, —OH, —CN and —NO₂; wherein each heteroaryl or heterocyclyl comprises 1 to 4 heteroatoms, independently selected from the group consisting of N, O and S.

Each of the symbols R^{3a}, R^{3b}, R^{4a}, R^{4b}, R^{4c}, R^{11a}, R^{11b}, R^{11c}, R^{11d}, R^{11e}, R^{11f}, R^{12a}, R^{12b}, R^{12c} and R^{12d} are members independently selected from the group consisting of: H, C₁₋₆alkyl, C₂₋₆alkenyl, C₂₋₆alkynyl, C₀₋₄alkylC₃₋₈cycloalkyl, C₀₋₄alkylaryl, C₀₋₄alkyl-heteroaryl, —C₀₋₆alkyl-COC₁₋₄alkyl, —C₀₋₆alkyl-CO₂C₁₋₄alkyl, —C₀₋₆alkyl-SO₂—C₁₋₄alkyl, —C₀₋₆alkyl-SO₂—N(C₁₋₄alkyl, C₁₋₄alkyl), —C₀₋₆alkyl-N(C₁₋₄alkyl, C₁₋₄alkyl) and —C₁₋₆alkyl-O—C₀₋₆alkyl, wherein 1-3 hydrogen atoms on the aryl or heteroaryl ring may be independently replaced with a member selected from the group consisting of halo, C₁₋₆alkyl, C₂₋₆alkenyl, C₂₋₆alkynyl, C₀₋₄alkylC₃₋₈cycloalkyl, C₁₋₄alkoxy, —CO₂H, —CO₂C₁₋₄alkyl, —CON(C₁₋₄alkyl, C₁₋₄alkyl), —OH, —CN and NO₂; or can be taken together with the nitrogen atom to which they are attached to form a 3-8 membered heterocyclyl group, comprising 1 to 4 heteroatoms selected from the group consisting of N, O and S, each of which is optionally substituted with 1 to 4 R¹³ substituents selected from the group consisting of halo, C₁₋₆alkyl, C₂₋₆alkenyl, C₂₋₆alkynyl, C₀₋₄alkylC₃₋₈cycloalkyl, C₁₋₄alkoxy, —CO₂H, —CO₂C₁₋₄alkyl, —CON(C₁₋₄alkyl, C₁₋₄alkyl), ═O, ═S, —OH, —CN and NO₂.

Each of the symbols R⁶, R⁷, R⁸, R^{10a} and R^{10b} is a member independently selected from the group consisting of: hydrogen, C₁₋₆alkyl, C₂₋₆alkenyl, C₂₋₆alkynyl and C₀₋₄alkylC₃₋₈cycloalkyl, —C₀₋₆alkyl-aryl and —C₀₋₆alkyl-heteroaryl; or R⁴ and R⁶ can be taken together with the atoms to which they are attached to form a 5 to 12 membered heterocyclyl group; wherein each heterocyclyl is a 5 to 8 membered monocyclic ring or a 8-12 membered bicyclic ring, each comprising from 1 to 4 heteroatoms selected from the group consisting of O, N and S, and wherein 1 to 3 carbon or nitrogen atoms of aryl, heteroaryl and heterocyclyl are substituted with 1 to 3 R^{4d} substituents.

Each subscript n1 and n2 represents an integer of 0 to 1.

The present invention further provides chemical intermediates, pharmaceutical compositions and methods for preventing or treating a condition in a mammal characterized by undesired thrombosis comprising the step of administering to said mammal a therapeutically effective amount of a compound of the present invention. Such conditions include but are not limited to acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, conditions requiring the fitting of prosthetic devices, and the like.

The present invention further provides methods for inhibiting the coagulation of a blood sample comprising contacting said sample with a compound of the present invention.

### BRIEF DESCRIPTION OF THE DRAWINGS

FIG. 1 illustrates a variety of embodiments of compounds of the invention.

### DETAILED DESCRIPTION OF THE INVENTION

Abbreviations and Definitions

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group is one having one or more double bonds. Similarly, the term “alkynyl” refers to an unsaturated alkyl group having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “cycloalkyl” refers to hydrocarbon rings having the indicated number of ring atoms (e.g., C₃₋₈cycloalkyl) and being fully saturated or having no more than one double bond between ring vertices. When “cycloalkyl” is used in combination with “alkyl”, as in C₃₋₅cycloalkyl-alkyl, the cycloalkyl portion is meant to have from three to five carbon atoms, while the alkyl portion is an alkylene moiety having from one to three carbon atoms (e.g., —CH₂—, —CH₂CH₂— or —CH₂CH₂CH₂—).

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having four or fewer carbon atoms.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups (typically provided as —NR^{a}R^{b} or a variant thereof), the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “aryl” means, unless otherwise stated, a polyunsaturated, typically aromatic, hydrocarbon group which can be a single ring or multiple rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom or through a carbon atom. Non-limiting examples of aryl groups include phenyl, naphthyl and biphenyl, while non-limiting examples of heteroaryl groups include 1-pyrrolyl, 2-pyrrolyl, 3-pyrrolyl, 1-pyrazolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, benzopyrazolyl, 5-indolyl, 1-isoquinolyl, 5-isoquinolyl, 2-quinoxalinyl, 5-quinoxalinyl, 3-quinolyl, and 6-quinolyl. If not specifically stated, substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like).

The terms “heterocycle” and “heterocyclyl” refers to a saturated or unsaturated non-aromatic cyclic group containing at least one sulfur, nitrogen or oxygen heteroatom. Each heterocycle can be attached at any available ring carbon or heteroatom. Each heterocycle may have one (“heteromonocyclyl”) or more rings (e.g. “heterobicyclyl”). When multiple rings are present, they can be fused together or linked covalently. Each heterocycle must contain at least one heteroatom (typically 1 to 5 heteroatoms) selected from nitrogen, oxygen or sulfur. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Non-limiting examples of heterocycle groups include pyrrolidine, piperidine, imidazolidine, pyrazolidine, butyrolactam, valerolactam, imidazolidinone, hydantoin, dioxolane, phthalimide, 1,4-dioxane, morpholine, thiomorpholine, thiomorpholine-S,S-dioxide, piperazine, pyran, pyridone, 3-pyrroline, thiopyran, pyrone, tetrahydrofuran, tetrahydrothiophene and the like.

The above terms (e.g., “aryl” and “heteroaryl”), in some embodiments, will include both substituted and unsubstituted forms of the indicated radical. Preferred substituents for each type of radical are provided below. For brevity, the terms aryl and heteroaryl will refer to substituted or unsubstituted versions as provided below.

Substituents for the aryl and heteroaryl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″CO₂R′, —NR′—C(O)NR″R′″, —NH—C(NH₂)═NH, —NR′C(NH₂)═NH, —NH—C(NH₂)═NR′, —S(O)R′, —SO₂R′, —SO₂NR′R″, —NR′SO₂R″, —N₃, perfluoro(C₁-C₄)alkoxy, and perfluoro(C₁-C₄)alkyl, in a number ranging from zero to the total number of open valences on the aromatic ring system; and where R′, R″ and R′″ is independently selected from hydrogen, C₁₋₆alkyl, C₃₋₈cycloalkyl, C₂₋₆alkenyl, C₂₋₆alkynyl, unsubstituted aryl and heteroaryl, (unsubstituted aryl)-C₁₋₄alkyl, and unsubstituted aryloxy-C₁₋₄alkyl. Other suitable substituents include each of the above aryl substituents attached to a ring atom by an alkylene tether of from 1-4 carbon atoms.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

General

### Embodiments of the Invention

Compounds

The present invention provides in one aspect, compounds having the formula: 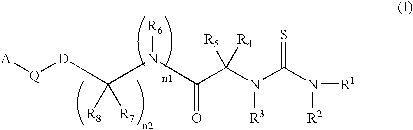
and pharmaceutically acceptable salts, hydrates, solvates and prodrugs thereof. In formula (I), the symbol R¹ represents a member selected from the group consisting of: hydrogen, —C₁₋₆alkyl, —C₀₋₆alkyl-aryl, heteroaryl and —C₂₋₆alkenyl.

The symbol R² represents a member selected from the group consisting of: a member selected from the group consisting of: —C₀₋₆alkyl-aryl, —C₃₋₈cycloalkylaryl, heteroaryl, heteroaryl-C₃₋₈cycloalkyl, —C₃₋₈cycloalkyl, —C₃₋₈cycloalkenyl, heteromonocyclyl, fused heterobicyclyl and unfused heterobicyclyl, each of which is optionally substituted with from 1 to 3 R^{2a} substituents, wherein each heteromonocyclyl, fused heterobicyclyl or unfused heterobicyclyl comprises 5 to 12 ring atoms, 1 to 4 of which are members independently selected from the group consisting of N, O and S.

The symbol R³ represents a member selected from the group consisting of: hydrogen, C₁₋₆alkyl, heteroaryl, C₂₋₆alkenyl, —C₀₋₄alkyl-C₃₋₈-cycloalkyl, —C₀₋₆alkyl-aryl, —C₀₋₆alkyl-heteroaryl, —C₀₋₆alkyl-heterocyclyl, —C₀₋₆alkyl-CO—OR^{3a}, —C₁₋₆alkyl-N(R^{3a}R^{3b}), —C₁₋₆alkyl-O—R^{3a}, —C₁₋₆alkyl-S—R^{3a}, —C₀₋₆alkyl-C(O)N(R^{3a}R^{3b}) and —C₁₋₆alkyl-N(R^{3a})C(O)R^{3b}.

Each R⁴ and R⁵ is a member independently selected from the group consisting of: hydrogen, —C₁₋₆alkyl, —C₂₋₆alkenyl, —C₂₋₆alkynyl, —C₀₋₄alkyl-C₃₋₈-cycloalkyl, C₁₋₆haloalkyl, —C₀₋₆alkyl-heteroaryl, —C₀₋₆alkyl-heterocyclyl, —C₀₋₆alkyl-CN, —C₀₋₆alkyl-NO₂, —C₁₋₆alkyl-O—R^{4a}, —C₁₋₆alkyl-S—R^{4a}, —C₁₋₆alkyl-SO₂—R^{4a}, —C₁₋₆alkyl-S(O)—R^{4a}, —C₀₋₆alkyl-CO—OR^{4a}, —C₀₋₆alkyl-C(O)—N(R^{4a}R^{4b}), —C₀₋₆alkyl-C(O)R^{4a}, —C₁₋₆alkyl-N(R^{4a}R^{4b}), —C₁₋₆alkyl-N(R^{4a})R^{4b}, —C₁₋₆alkyl-N(R^{4a})—C(O)—N(R^{4b}R^{4c}), —C₁₋₆alkyl-N(R^{4a})—SO₂—R^{4b}, —C₁₋₆alkyl-SO₂—N(R^{4a}R^{4b}), —C₀₋₆alkyl-PO(—OR^{4a})(OR^{4b}), —C₁₋₆alkyl-N(R^{4a})—PO(—OR^{4b})(—OR^{4c}), —C₀₋₆alkyl-aryl, —C₀₋₆alkyl-heteroaryl, and —C₀₋₆alkyl-heterocyclyl; or R⁴ and R⁵ can be taken together with the carbon atom to which they are attached to form a 3 to 8 membered heterocyclyl group; wherein each heterocyclyl is a 3 to 8 membered monocyclic ring or a 8-12 membered bicyclic ring, each comprising from 1 to 4 heteroatoms selected from the group consisting of O, N and S, and wherein 1 to 3 carbon or nitrogen atoms of aryl, heteroaryl and heterocyclyl are substituted with 1 to 3 R^{4d} substituents.

The letter D is a member selected from the group consisting of: a direct bond, aryl, heteroaryl, C₃₋₈cycloalkyl, C₃₋₈cycloalkylene, heteromonocyclyl, unfused heterobicyclyl, and fused heterobicyclyl; each of which is optionally substituted with 1 to 3 R⁹ substituents, wherein each heteromonocyclyl, fused heterobicyclyl or unfused heterobicyclyl comprises from 5 to 10 ring atoms, 1-4 of which are selected from the group consisting of N, O and S.

The symbol Q is selected from the group consisting of: a direct bond, —C(R^{10a}R^{10b})—, —C(O)—, —C(S)—, C(═NR^{10a})—, —O—, —S—, —N(R^{10a}), —N(R^{10a})CH₂—, —CH₂N(R^{10a}), —C(O)N(R^{10a})—, —N(R^{10a})C(O)—, —SO₂—, —SO—, —SO₂N(R^{10a})—, and —N(R^{10a})—SO₂—; and at least one of D and Q is not a direct bond.

The symbol A is selected from the group consisting of: —NR^{11c}R^{11d}, —C(═NR^{11c})NR^{11a}R^{11b}, —C(═NR^{11e}R^{11f})NR^{11a}R^{11b}, —N(R^{11d})C(═NR^{11c})NR^{11a}R^{11b}, —N(R^{11d})C(═NR^{11c})R^{11a}, —N(R^{11c})NR^{11a}R^{11b}, —N(R^{11c})OR^{11d}; C₁₋₆alkyl, C₂₋₆alkenyl, aryl, heteroaryl, C₃₋₈cycloalkyl, C₃₋₈cycloalkenyl, heteromonocyclyl, and fused heterobicyclyl; each of aryl, heteroaryl, heteromonocyclyl and fused heterobicyclyl, each of which is optionally substituted with 1 to 3 R^{11g}; wherein each hetercyclyl comprises from 5 to 10 ring atoms, 1-4 of which are selected from the group consisting of N, O and S.

Each R^{2a}, R^{4d}, R⁹ and R^{11g} is a member independently selected from the group consisting of: H, halo, C₁₋₆alkyl, C₂₋₆alkenyl, C₂₋₆alkynyl, C₀₋₄alkylC₃₋₈cycloalkyl, —C₁₋₄alkoxy, —O—C₀₋₂alkyl-CF₃, —C₀₋₂alkyl-CF₃, —C₀₋₂alkyl-CN, —C₀₋₂alkyl-NO₂, —C₀₋₂alkyl-NR^{12a}R^{12b}, —C₀₋₂alkyl-SO₂NR^{12a}R^{12b}, —C₀₋₂alkyl-SO₂R^{12a}, —C₀₋₂alkyl-SOR^{12a}, —C₀₋₂alkyl-CF₃, —C₀₋₂alkyl-OR^{12a}, —C₀₋₂alkyl-SR^{12a}, —CH₂—CH₂—OR^{12a}, —O—CH₂—CO₂R^{12a}, —N(R^{12a})—CH₂—CH₂ OR^{12b}, —C₀₋₂alkyl-C(O)NR^{12a}R^{12b}, —C₀₋₂alkyl-CO₂R^{12a}, —C₀₋₂alkyl-N(R^{12a})—C(O)R^{12b}, —C₀₋₂alkyl-N(R^{12c})—C(O)NR^{12a}R^{12b}—C₀₋₂alkyl-C(═NR^{12c})NR^{12a}R^{12b}, —C₀₋₂alkyl-C(═NR^{12a})R^{12b}, —C₀₋₂alkyl-N(R^{12d})C(═NR^{12c})NR^{12a}R^{12b}, —C₀₋₂alkyl-N(R^{12a})—SO₂—R^{12b}, ═O, ═S, ═NR^{12a}, 5- or 6-membered aryl, 5- or 6-membered heteroaryl and 5- to 7-membered heterocyclyl, each of which is optionally substituted with a member independently selected from the group consisting of halo, CF₃, OCF₃, SCF₃, C₁₋₆alkyl, C₂₋₆alkenyl, C₂₋₆alkynyl, C₀₋₄alkylC₃₋₈cycloalkyl, C₁₋₄alkoxy, —CO₂H, —CO₂C₁₋₄alkyl, —CONR^{12a}R^{12b}, ═O, ═S, —OH, —CN and —NO₂; wherein each heteroaryl or heterocyclyl comprises 1 to 4 heteroatoms, independently selected from the group consisting of N, O and S.

Each of the symbols R^{3a}, R^{3b}, R^{4a}, R^{4b}, R^{4c}, R^{11a}, R^{11b}, R^{11c}, R^{11d}, R^{11e}, R^{11f}, R^{12a}, R^{12b}, R^{12c} and R^{12d} are members independently selected from the group consisting of: H, C₁₋₆alkyl, C₂₋₆alkenyl, C₂₋₆alkynyl, C₀₋₄alkylC₃₋₈cycloalkyl, C₀₋₄alkylaryl, C₀₋₄alkyl-heteroaryl, —C₀₋₆alkyl-COC₄alkyl, —C₀₋₆alkyl-CO₂C₁₋₄alkyl, —C₀₋₆alkyl-SO₂—C₁₋₄alkyl, —C₀₋₆alkyl-SO₂—N(C₁₋₄alkyl, C₁₋₄alkyl), —C₀₋₆alkyl-N(C₁₋₄alkyl, C₁₋₄alkyl) and —C₁₋₆alkyl-O—C₀₋₆alkyl, wherein 1-3 hydrogen atoms on the aryl or heteroaryl ring may be independently replaced with a member selected from the group consisting of halo, C₁₋₆alkyl, C₂₋₆alkenyl, C₂₋₆alkynyl, C₀₋₄alkylC₃₋₈cycloalkyl, C₁₋₄alkoxy, —CO₂H, —CO₂C₁₋₄alkyl, —CON(C₁₋₄alkyl, C₁₋₄alkyl), —OH, —CN and NO₂; or can be taken together with the nitrogen atom to which they are attached to form a 3-8 membered heterocyclyl group, comprising 1 to 4 heteroatoms selected from the group consisting of N, O and S, each of which is optionally substituted with 1 to 4 R¹³ substituents selected from the group consisting of halo, C₁₋₆alkyl, C₂₋₆alkenyl, C₂₋₆alkynyl, C₀₋₄alkylC₃₋₈cycloalkyl, C₁₋₄alkoxy, —CO₂H, —CO₂C₁₋₄alkyl, —CON(C₁₋₄alkyl, C₁₋₄alkyl), ═O, ═S, —OH, —CN and NO₂.

Each of the symbols R⁶, R⁷, R⁸, R^{10a} and R^{10b} is a member independently selected from the group consisting of: hydrogen, C₁₋₆alkyl, C₂₋₆alkenyl, C₂₋₆alkynyl and C₀₋₄alkylC₃₋₈cycloalkyl, —C₀₋₆alkyl-aryl and —C₀₋₆alkyl-heteroaryl; or R⁴ and R⁶ can be taken together with the atoms to which they are attached to form a 5 to 12 membered heterocyclyl group; wherein each heterocyclyl is a 5 to 8 membered monocyclic ring or a 8-12 membered bicyclic ring, each comprising from 1 to 4 heteroatoms selected from the group consisting of O, N and S, and wherein 1 to 3 carbon or nitrogen atoms of aryl, heteroaryl and heterocyclyl are substituted with 1 to 3 R^{4d} substituents. Each subscript n1 and n2 represents an integer of 0 to 1.

With the above formula are a number of specific embodiments of the invention. In one group of embodiments, R¹ and R³ is H. In a specific group of embodiments, R² is aryl or heteroaryl, each of which is optionally substituted with 1 to 3 R^{2a}. More preferably, R² is selected from the group consisting of phenyl, pyridyl and benzofuranyl. More preferably, R² is independently selected from the group consisting of halo, C₁₋₆alkyl, C₂₋₆alkynyl, —C₁₋₄alkoxy, —O—C₀₋₂alkyl-CF₃ and —C₀₋₂alkyl-CF₃. For these embodiments, a preferred group of embodiments are those in which R^{2a} is attached to the phenyl or pyridyl ring at a position para to the rest of the molecule.

In one group of embodiments, R⁴ and R⁵ is a member independently selected from the group consisting of: hydrogen, —C₁₋₆alkyl and —C₀₋₆alkyl-aryl. More preferably R⁴ is hydrogen and R⁵ is a member independently selected from the group consisting of hydrogen, isopropyl, isobutyl and phenyl. In a specific group of embodiments when R⁴ and R⁵ are different, the carbon bearing R⁵ has the R-configuration. In another group of embodiments when R⁴ and R⁵ are different, the carbon bearing R⁵ has the S-configuration.

In one group of embodiments, the subscript n1 is 0. In another group of embodiments the subscript n1 is 1. In a specific group of embodiments R⁶ is H or R⁴ and R⁶ can be taken together with the atoms to which they are attached to form a 5 to 12 membered heterocyclyl group; wherein each heterocyclyl is a 5 to 8 membered monocyclic ring or a 8-12 membered bicyclic ring, each comprising from 1 to 4 heteroatoms selected from the group consisting of O, N and S, and wherein 1 to 3 carbon or nitrogen atoms of aryl, heteroaryl and heterocyclyl are substituted with 1 to 3 R^{4d} substituents. In one group of embodiments, R⁴ and R⁶ are taken together with the atoms to which they are attached selected from the group having the formula: 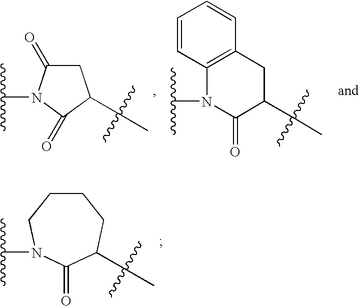
optionally substituted with 1 to 3 R^{4d} substituents. In another group of embodiments, the subscript n2 is 0. In another group of embodiments the subscript n2 is 1.

In one group of embodiments, D is aryl or heteromonocyclyl, wherein each heterocyclyl comprises from 5 to 7 ring atoms, 1 to 2 of which are N or O. More preferably, D is phenyl, piperazinyl or piperidinyl. In another group of embodiments, Q is a direct bond or —C(═NH)— More preferably, Q is attached to the phenyl or piperazinyl ring at a position para to the rest of the molecule.

In another group of embodiments, A is selected from the group consisting of: —NR^{11a}R^{11b}, aryl, heteroaryl and heteromonocyclyl; each of aryl, heteroaryl, heteromonocyclyl and fused heterobicyclyl, each of which is optionally substituted with 1 to 3 R^{11g}; wherein each hetercyclyl comprises from 5 to 7 ring atoms, 1 to 2 of which are selected from the group consisting of N and O. More preferably, A is a member selected from the group consisting of pyridinyl, dihydroimidazolyl, pyrrolidinyl, azetidinyl, piperidinyl, homopiperidinyl, morpholinyl and phenyl. Other embodiments are in which each optional substituent R^{11g} is independently selected from the group consisting of halo, C₁₋₆alkyl, C₂₋₆alkynyl, —O—C₀₋₂alkyl-CF₃, —C₀₋₂alkyl-CF₃ and ═O. Other embodiments are wherein A-Q-D-(CR⁷R⁸)ₙ₂—NR⁶ₙ₁ is selected from the group consisting of: 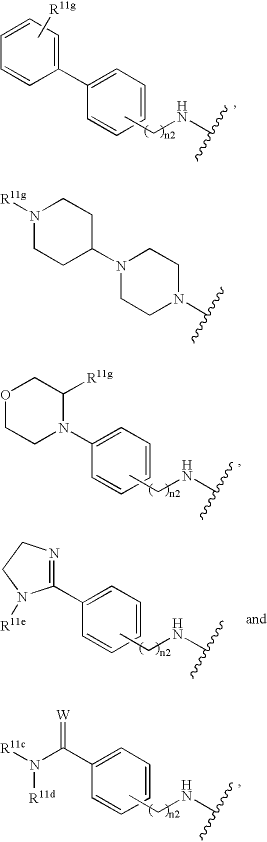
wherein W is O, S or NH; and the wavy line indicates the point of attachment to the rest of the molecule. Other embodiments are wherein A-Q-D-(CR⁷R⁸)ₙ₂—NR⁶ₙ₁ is selected from the group consisting of:

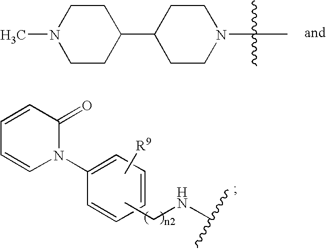

wherein the wavy line indicates the point of attachment to the rest of the molecule. Other embodiments are wherein A-Q- is selected from the group consisting of:

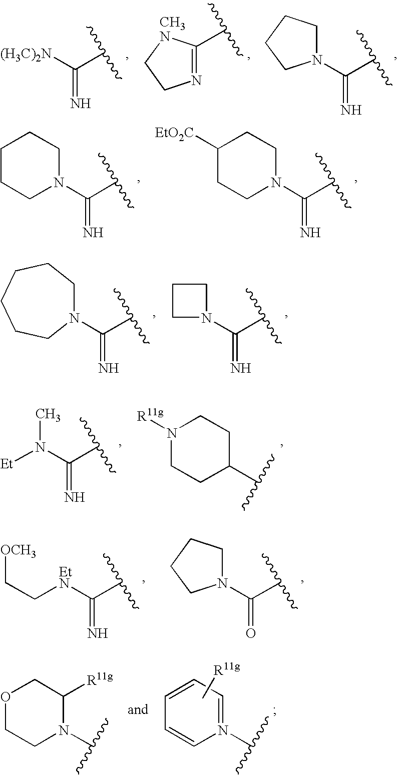

wherein the wavy line indicates the point of attachment to the rest of the molecule. Other embodiments are wherein A-Q- is selected from the group consisting of: 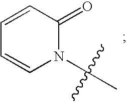
wherein the wavy line indicates the point of attachment to the rest of the molecule.

In other embodiments, compounds of formula I are provided which have the formula: 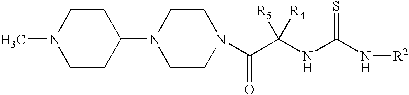
wherein R² is aryl, optionally substituted with 1 to 3 R^{2a}. Within this group, specific embodiments are provided in which R² is phenyl. Preferably, the optional substituent R^{2a} is halo. Still further preferred are embodiments, wherein R^{2a} is attached to the phenyl ring at a position para to the rest of the molecule. Yet another group of embodiments are those in which each R⁴ and R⁵ is a member independently selected from the group consisting of: hydrogen and —C₀₋₆alkyl-aryl. More preferably R⁴ is hydrogen and R⁵ is phenyl. In a specific group of embodiments when R⁴ and R⁵ are different, the carbon bearing R⁵ has the R-configuration. In another group of embodiments when R⁴ and R⁵ are different, the carbon bearing R⁵ has the S-configuration.

In other embodiments, compounds of formula I are provided which have the formula: 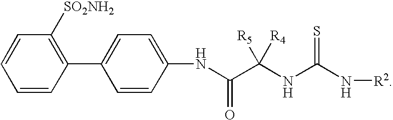
Within this group, specific embodiments are provided in which R² is aryl or heteroaryl, each of which is optionally substituted with 1 to 3 R^{2a}. Preferably, R² is selected from the group consisting of phenyl. Still further preferred are embodiments wherein each optional substituent R^{2a} is independently selected from the group consisting of halo and C₁₋₆alkyl. Still further preferred are embodiments wherein R^{2a} is attached to the phenyl ring at a position para to the rest of the molecule. Yet another group of embodiments are those in which each R⁴ and R⁵ is a member independently selected from the group consisting of: hydrogen, —C₁₋₆alkyl and —C₀₋₆alkyl-aryl. More preferably R⁴ is hydrogen and R⁵ is a member independently selected from the group consisting of hydrogen, isobutyl and phenyl. In a specific group of embodiments when R⁴ and R⁵ are different, the carbon bearing R⁵ has the R-configuration. In another group of embodiments when R⁴ and R⁵ are different, the carbon bearing R⁵ has the S-configuration.

In other embodiments, compounds of formula I are provided which have the formula: 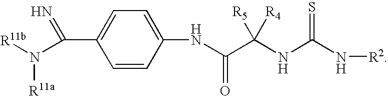
Within this group, specific embodiments are provided in which R² is aryl or heteroaryl, each of which is optionally substituted with 1 to 3 R^{2a}. Preferably, R² is selected from the group consisting of phenyl and benzofuranyl. Still further preferred are embodiments wherein each optional substituent R^{2a} is independently selected from the group consisting of halo and C₁₋₆alkyl. Still further preferred are embodiments wherein R^{2a} is attached to the phenyl ring at a position para to the rest of the molecule. Yet another group of embodiments are those in which each R⁴ and R⁵ is a member independently selected from the group consisting of: hydrogen, —C₁₋₆alkyl and —C₀₋₆alkyl-aryl. More preferably R⁴ is hydrogen and R⁵ is a member independently selected from the group consisting of hydrogen, isobutyl and phenyl. In a specific group of embodiments when R⁴ and R⁵ are different, the carbon bearing R⁵ has the R-configuration. In another group of embodiments when R⁴ and R⁵ are different, the carbon bearing R⁵ has the S-configuration.

In other embodiments, compounds are provided in which each R^{11a} and R^{11b} is C₁₋₆alkyl.

In other embodiments, compounds of formula I are provided which have the formula: 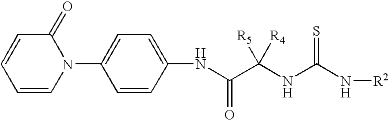
Within this group, specific embodiments are provided in which R² is aryl or heteroaryl, each of which is optionally substituted with 1 to 3 R^{2a}. Preferably, R² is selected from the group consisting of phenyl and benzofuranyl. Still further preferred are embodiments wherein each optional substituent R^{2a} is independently selected from the group consisting of halo and C₁₋₆alkyl. Still further preferred are embodiments wherein R^{2a} is attached to the phenyl ring at a position para to the rest of the molecule. Yet another group of embodiments are those in which each R⁴ and R⁵ is a member independently selected from the group consisting of: hydrogen, —C₁₋₆alkyl and —C₀₋₆alkyl-aryl. More preferably R⁴ is hydrogen and R⁵ is a member independently selected from the group consisting of hydrogen, isobutyl and phenyl. In a specific group of embodiments when R⁴ and R⁵ are different, the carbon bearing R⁵ has the R-configuration. In another group of embodiments when R⁴ and R⁵ are different, the carbon bearing R⁵ has the S-configuration.

In each of the above embodiments, any variables are meant to have their full scope w/ reference to formula (I) unless indicated otherwise.

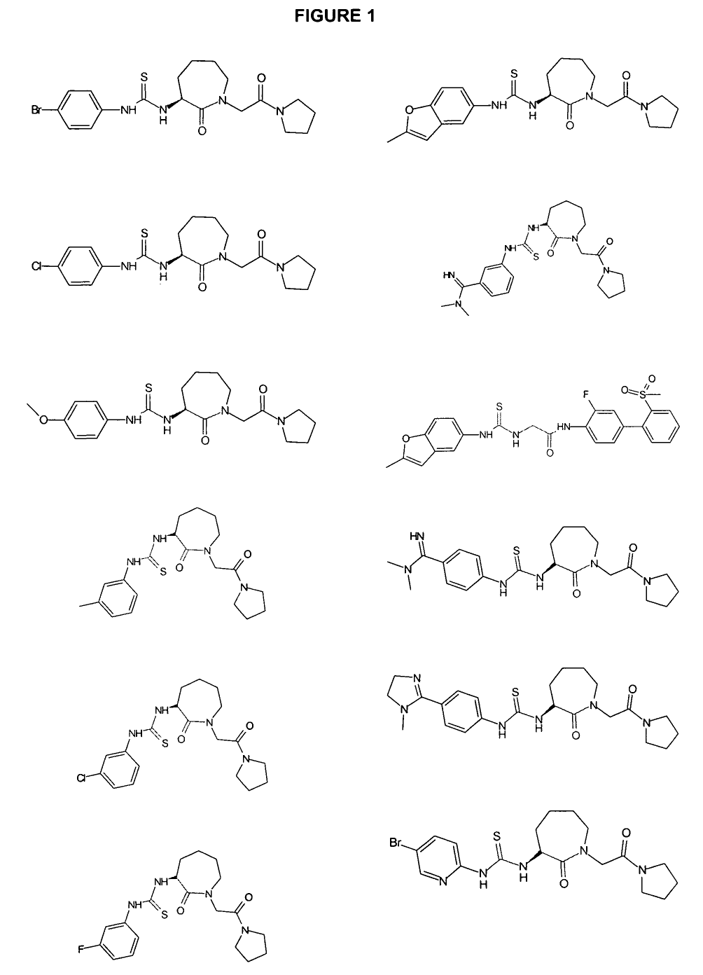

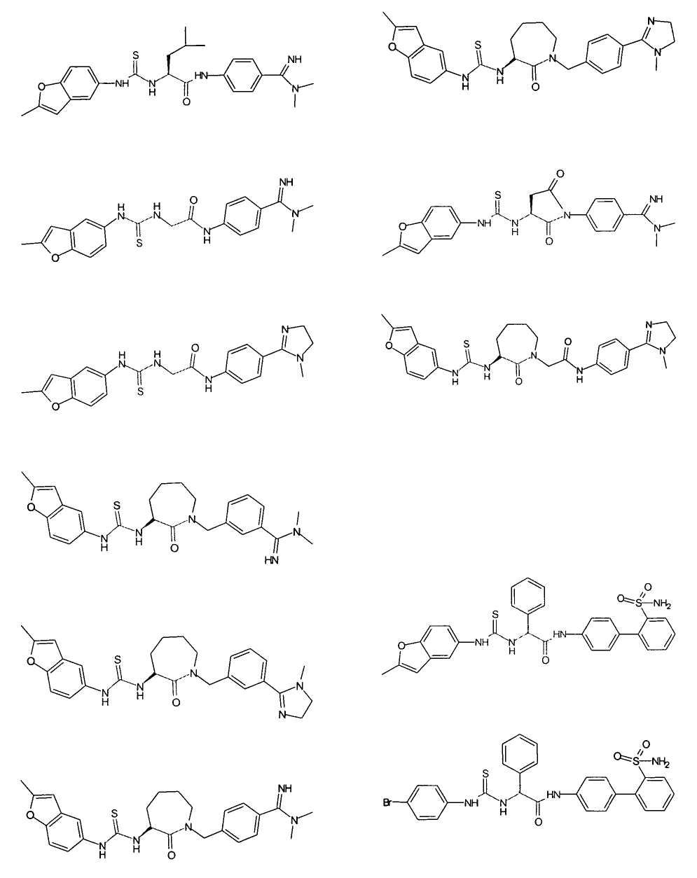

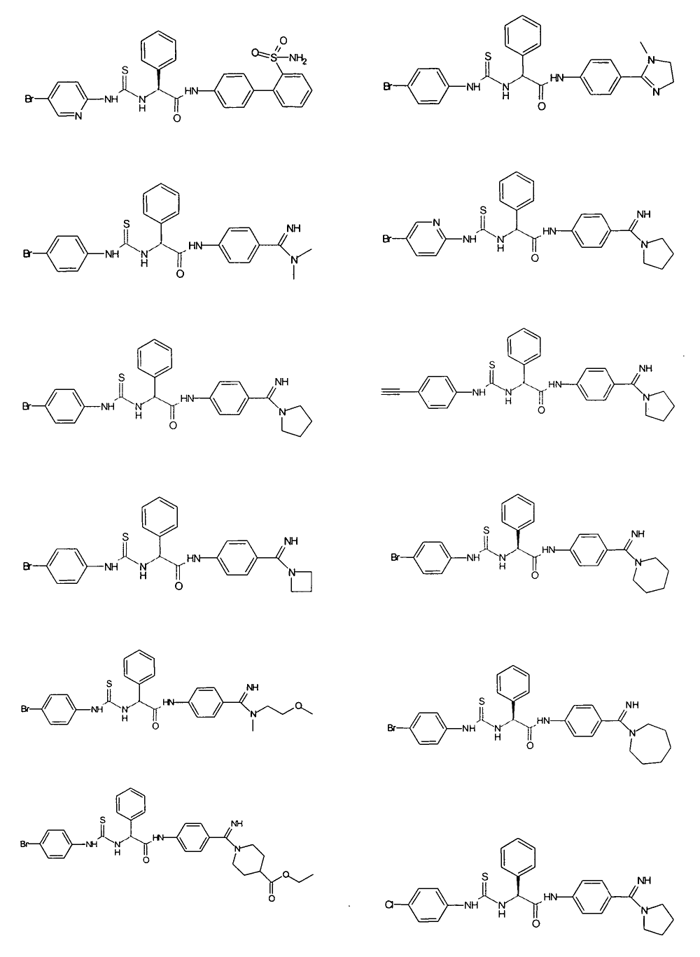

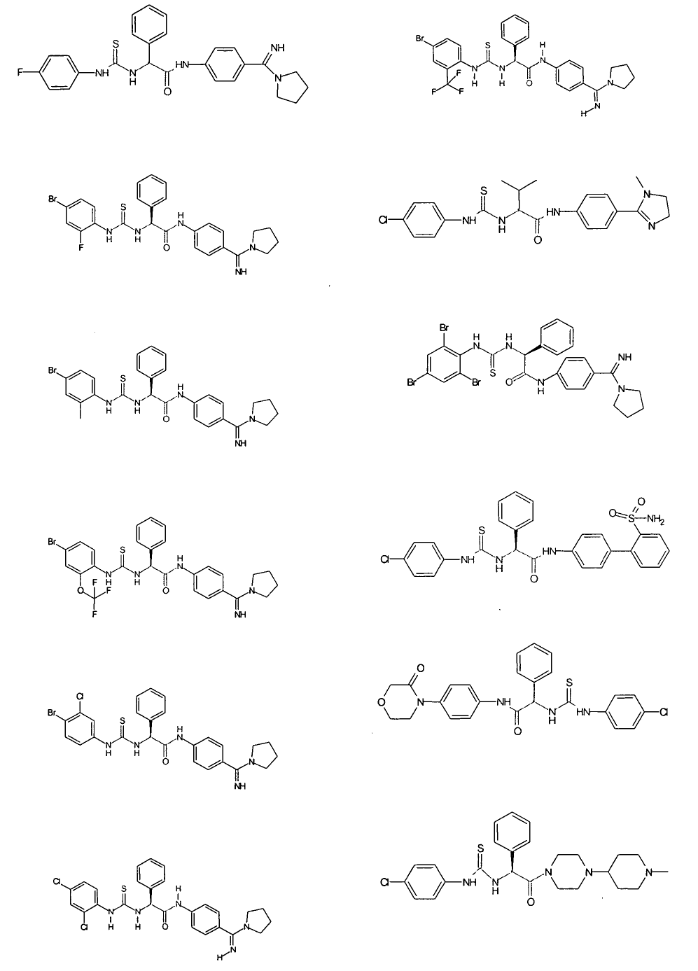

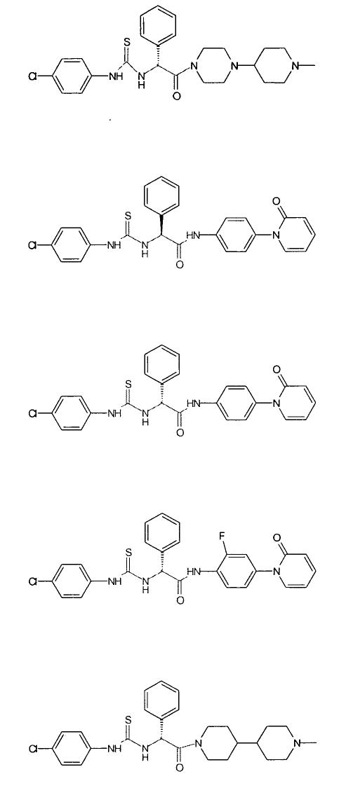

A variety of compounds have the desired activity. In particular, various embodiments of compounds are provided in FIG. 1.

Within the present invention, the compounds provided in the examples and figures are each preferred embodiments, along with their pharmaceutically acceptable salts, hydrates, solvates, and prodrugs thereof. Preferred examples of compounds of formula (I) include:
N-[4-(dimethylaminoimino)phenyl]-2-[(2-methylbenzo[b]furan-5-yl)aminothiocarbonylamino]-acetamide;
N-[4-(1-methyl-4,5-dihydro-1H-imidazol-2-yl)phenyl]-2-[(2-methylbenzo[b]furan-5-yl)aminothiocarbonylamino]-acetamide;
N-[4-(1-methyl-4,5-dihydro-1H-imidazol-2-yl)phenyl]-2-isobutyl-2-[(2-methylbenzo[b]furan-5-yl)aminothiocarbonylamino]-acetamide;
N-[4-(dimethylaminoimino)phenyl]-2-phenyl-2-(4-bromophenylaminothiocarbonylamino)-acetamide;
N-[4-(pyrrolidinylimino)phenyl]-2-phenyl-2-(4-bromophenylaminothiocarbonylamino)-acetamide;
N-[4-(azetidinylimino)phenyl]-2-phenyl-2-(4-bromophenylaminothiocarbonylamino)-acetamide;
N-{4-[(N-methyl-N-2-methoxyethyl)aminoimino]phenyl}-2-phenyl-2-(4-bromophenylaminothiocarbonylamino)-acetamide;
N-[4-(4-ethoxycarbonylpiperidinylimino)phenyl]-2-phenyl-2-(4-bromophenylaminothiocarbonylamino)-acetamide;
N-[4-(1-methyl-4,5-dihydro-1H-imidazol-2-yl)phenyl]-2-phenyl-2-(4-bromophenylaminothiocarbonylamino)-acetamide;
N-[4-(pyrrolidinylimino)phenyl]-2-phenyl-2-(4-fluorophenylaminothiocarbonylamino)-acetamide;
N-[4-(pyrrolidinylimino)phenyl]-2-phenyl-2-(4-ethynylphenylaminothiocarbonylamino)-acetamide;
N-[4-(pyrrolidinylimino)phenyl]-2-phenyl-2-(5-bromopyridin-2-ylaminothiocarbonylamino)-acetamide;
(S) N-[4-(pyrrolidinylimino)phenyl]-2-phenyl-2-(4-bromophenylaminothiocarbonylamino)-acetamide;
(S) N-[4-(piperidinylimino)phenyl]-2-phenyl-2-(4-bromophenylaminothiocarbonylamino)-acetamide;
(S) N-[4-(homopiperidinylimino)phenyl]-2-phenyl-2-(4-bromophenylaminothiocarbonylamino)-acetamide;
(S) N-[4-(pyrrolidinylimino)phenyl]-2-phenyl-2-(4-chlorophenylaminothiocarbonylamino)-acetamide;
(S) N-[4-(pyrrolidinylimino)phenyl]-2-phenyl-2-(2-fluoro-4-bromophenylaminothiocarbonylamino)-acetamide;
(S) N-[4-(pyrrolidinylimino)phenyl]-2-phenyl-2-(2-trifluoromethoxy-4-bromophenylaminothiocarbonylamino)-acetamide;
(S) N-[4-(pyrrolidinylimino)phenyl]-2-phenyl-2-(2-methyl-4-bromophenylaminothiocarbonylamino)-acetamide;
(S) N-[4-(pyrrolidinylimino)phenyl]-2-phenyl-2-(2,4-dichlorophenylaminothiocarbonylamino)-acetamide;
(S) N-[4-(pyrrolidinylimino)phenyl]-2-phenyl-2-(3-chloro-4-bromophenylaminothiocarbonylamino)-acetamide;
(S) N-[4-(pyrrolidinylimino)phenyl]-2-phenyl-2-(2-trifluoromethyl-4-bromophenylaminothiocarbonylamino)-acetamide;
(S) N-[4-(pyrrolidinylimino)phenyl]-2-phenyl-2-(2,4,6-tribromophenylaminothiocarbonylamino)-acetamide;
N-[4-(1-methyl-4,5-dihydro-1H-imidazol-2-yl)phenyl]-2-isopropyl-2-(4-chlorophenylaminothiocarbonylamino]-acetamide;
N-[4-(3-oxo-morpholin-4-yl)phenyl]-2-phenyl-2-(4-chlorophenylaminothiocarbonylamino)-acetamide;
(S) N-[4-(1-methylpiperidin-4-yl)piperazin-1-yl]-2-phenyl-2-(4-chlorophenylaminothiocarbonylamino)-acetamide;
(R) N-[4-(1-methylpiperidin-4-yl)piperazin-1-yl]-2-phenyl-2-(4-chlorophenylaminothiocarbonylamino)-acetamide;
N-[4-(2-aminosulfonylphenyl)phenyl]-2-phenyl-2-[(2-methylbenzo[b]furan-5-yl)aminothiocarbonylamino]-acetamide;
N-[4-(2-aminosulfonylphenyl)phenyl]-2-phenyl-2-[(4-bromophenyl)aminothiocarbonylamino]-acetamide;
(S) N-[4-(2-aminosulfonylphenyl)phenyl]-2-phenyl-2-(5-bromopyridin-2-ylaminothiocarbonylamino)-acetamide;
(R) N-[4-(2-aminosulfonylphenyl)phenyl]-2-phenyl-2-(5-bromopyridin-2-ylaminothiocarbonylamino)-acetamide;
(S) N-[4-(2-aminosulfonylphenyl)phenyl]-2-phenyl-2-(4-chlorophenylaminothiocarbonylamino)-acetamide;
(R) N-[4-(2-aminosulfonylphenyl)phenyl]-2-phenyl-2-(4-chlorophenylaminothiocarbonylamino)-acetamide;
(2S) N-[4-(2-pyridon-1-yl)phenyl]-2-phenyl-2-(4-chlorophenylaminothiocarbonylamino)-acetamide;
(2R) N-[4-(2-pyridon-1-yl)phenyl]-2-phenyl-2-(4-chlorophenylaminothiocarbonylamino)-acetamide;
(2R) N-[4-(2-pyridon-1-yl)-2-fluorophenyl]-2-phenyl-2-(4-chlorophenylaminothiocarbonylamino)-acetamide;
(2R) N-[4-(2-pyridon-1-yl)-2-fluorophenyl]-2-phenyl-2-(4-chlorophenylaminothiocarbonylamino)-acetamide; and
(2R) 4-(1-methylpiperidin-4-yl)piperidin-1-yl-2-phenyl-2-(4-chlorophenylaminothiocarbonylamino)-acetamide.

All the preferred, more preferred, and most preferred compounds listed above are selective inhibitors of Factor Xa.

Compositions

The present invention further provides compositions comprising one or more compounds of formula (I) or a tautomer or a pharmaceutically acceptable salt thereof, and a pharmaceutically acceptable carrier. It will be appreciated that the compounds of formula (I) in this invention may be derivatized at functional groups to provide prodrug derivatives which are capable of conversion back to the parent compounds in vivo. Examples of such prodrugs include the physiologically acceptable and metabolically labile ester derivatives, such as methoxymethyl esters, methylthiomethyl esters, or pivaloyloxymethyl esters derived from a hydroxyl group of the compound or a carbamoyl moiety derived from an amino group of the compound. Additionally, any physiologically acceptable equivalents of the compounds of formula (I), similar to metabolically labile esters or carbamates, which are capable of producing the parent compounds of formula (I) in vivo, are within the scope of this invention.

If pharmaceutically acceptable salts of the compounds of this invention are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Furthermore, the basic nitrogen-containing groups may be quaternized with agents like lower alkyl halides, such as methyl, ethyl, propyl and butyl chlorides, bromides and iodides; dialkyl sulfates, such as dimethyl, diethyl, dibutyl and diamyl sulfates, long chain halides, such as decyl, lauryl, myristyl and stearyl chlorides, bromides and iodides; aralkyl halides, such as benzyl and phenethyl bromides and others. Water or oil-soluble or dispersible products are thereby obtained.

The compounds utilized in the compositions and methods of this invention may also be modified by appending appropriate functionalities to enhance selective biological properties. Such modifications are known in the art and include those which increase biological penetration into a given biological system (e.g., blood, lymphatic system, central nervous system, etc.), increase oral availability, increase solubility to allow administration by injection, alter metabolism and alter rate of excretion.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

Pharmaceutically acceptable carriers that may be used in these compositions include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to a preferred embodiment, the compositions of this invention are formulated for pharmaceutical administration to a mammal, preferably a human being. Such pharmaceutical compositions of the invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. The formulations of the invention may be designed as short-acting, fast-releasing, or long-acting. Still further, compounds can be administered in a local rather than systemic means, such as administration (e.g., injection) as a sustained release formulation.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

The pharmaceutical compositions of this invention may be in any orally acceptable dosage form, including capsules, tablets, aqueous suspensions or solutions. In the case of tablets for oral use, carriers that are commonly used include lactose and corn starch. Lubricating agents, such as magnesium stearate, are also typically added. For a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be in a topical form, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract may be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include mineral oil, sorbitan monostearate, polysorbate 60, cetyl esters, wax, cetyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative, such as benzylalkonium chloride. Alternatively, for ophthalmic uses, the pharmaceutical compositions may be formulated in an ointment, such as petrolatum.

The pharmaceutical compositions of this invention may also be administered by nasal aerosol or inhalation. Such compositions are prepared according to techniques known in the art of pharmaceutical formulation and may be prepared as solutions in saline, employing benzyl alcohol or other suitable preservatives, absorption promoters to enhance bioavailability, fluorocarbons and/or other conventional solubilizing or dispersing agents.

Any of the above dosage forms containing effective amounts are within the bounds of routine experimentation and within the scope of the invention. A therapeutically effective dose may vary depending upon the route of administration and dosage form. The preferred compound or compounds of the invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

Besides those representative dosage forms described above, pharmaceutically acceptable excipients and carriers and dosage forms are generally known to those skilled in the art and are included in the invention. It should be understood that a specific dosage and treatment regimen for any particular patient will depend upon a variety of factors, including the activity of the specific compound employed, the age, body weight, general health, sex and diet of the patient, and the time of administration, rate of excretion, drug combination, judgment of the treating physician and severity of the particular disease being treated. The amount of active ingredient(s) will also depend upon the particular compound and other therapeutic agent, if present, in the composition.

Methods of Use

The invention provides methods of inhibiting or decreasing Factor Xa activity as well as treating or ameliorating a Factor Xa associated state, symptom, disorder or disease in a patient in need thereof (e.g., human or non-human). “Treating” within the context of the invention means an alleviation of symptoms associated with a disorder or disease, or halt of further progression or worsening of those symptoms, or prevention or prophylaxis of the disease or disorder.

The term “mammal” includes organisms which express Factor Xa. Examples of mammals include mice, rats, cows, sheep, pigs, goats, horses, bears, monkeys, dogs, cats and, preferably, humans. Transgenic organisms which express Factor Xa are also included in this definition.

The inventive methods comprise administering an effective amount of a compound or composition described herein to a mammal or non-human animal. As used herein, “effective amount” of a compound or composition of the invention includes those amounts that antagonize or inhibit Factor Xa. An amount which antagonizes or inhibits Factor Xa is detectable, for example, by any assay capable of determining Factor Xa activity, including the one described below as an illustrative testing method. Effective amounts may also include those amounts which alleviate symptoms of a Factor Xa associated disorder treatable by inhibiting Factor Xa. Accordingly, “antagonists of Factor Xa” include compounds which interact with the Factor Xa and modulate, e.g., inhibit or decrease, the ability of a second compound, e.g., another Factor Xa ligand, to interact with the Factor Xa. The Factor Xa binding compounds are preferably antagonists of Factor Xa. The language “Factor Xa binding compound” (e.g., exhibits binding affinity to the receptor) includes those compounds which interact with Factor Xa resulting in modulation of the activity of Factor Xa. Factor Xa binding compounds may be identified using an in vitro (e.g., cell and non-cell based) or in vivo method. A description of an in vitro method is provided below.

The amount of compound present in the methods and compositions described herein should be sufficient to cause a detectable decrease in the severity of the disorder, as measured by any of the assays described in the examples. The amount of Factor Xa modulator needed will depend on the effectiveness of the modulator for the given cell type and the length of time required to treat the disorder. In certain embodiments, the compositions of this invention may further comprise another therapeutic agent. When a second agent is used, the second agent may be administered either as a separate dosage form or as part of a single dosage form with the compounds or compositions of this invention. While one or more of the inventive compounds can be used in an application of monotherapy to treat a disorder, disease or symptom, they also may be used in combination therapy, in which the use of an inventive compound or composition (therapeutic agent) is combined with the use of one or more other therapeutic agents for treating the same and/or other types of disorders, symptoms and diseases. Combination therapy includes administration of the two or more therapeutic agents concurrently or sequentially. The agents may be administered in any order. Alternatively, the multiple therapeutic agents can be combined into a single composition that can be administered to the patient. For instance, a single pharmaceutical composition could comprise the compound or pharmaceutically acceptable salt or solvate according to the formula I, another therapeutic agent (e.g., methotrexate) or a pharmaceutically acceptable salt or solvate thereof, and a pharmaceutically acceptable excipient or carrier.

The invention comprises a compound having the formula I, a method for making an inventive compound, a method for making a pharmaceutical composition from at least one inventive compound and at least one pharmaceutically acceptable carrier or excipient, and a method of using one or more inventive compounds to treat a variety of disorders, symptoms and diseases (e.g., inflammatory, autoimmune, neurological, neurodegenerative, oncology and cardiovascular), such as RA, osteoarthritis, irritable bowel disease IBD, asthma, chronic obstructive pulmonary disease COPD and MS. The inventive compounds and their pharmaceutically acceptable salts and/or neutral compositions may be formulated together with a pharmaceutically acceptable excipient or carrier and the resulting composition may be administered in vivo to mammals, such as men, women and animals, to treat a variety of disorders, symptoms and diseases. Furthermore, the inventive compounds can be used to prepare a medicament that is useful for treating a variety of disorders, symptoms and diseases.

Kits

Still another aspect of this invention is to provide a kit comprising separate containers in a single package, wherein the inventive pharmaceutical compounds, compositions and/or salts thereof are used in combination with pharmaceutically acceptable carriers to treat states, disorders, symptoms and diseases where Factor Xa plays a role.

### EXAMPLES

### Example 1

### N-[4-(dimethylaminoimino)phenyl]-2-[(2-methylbenzo[b]furan-5-yl)aminothiocarbonylamino]-acetamide

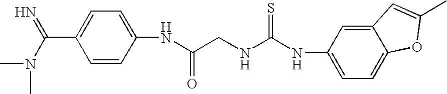

### A. Preparation of 2-methylbenzo[b]furan-5-yl isothiocyanate

To a suspension of NaH (60% in mineral oil, 5.54 g, 139 mmol) in THF (70 mL), a solution of acetone oxime (10.0 g, 137 mmol) in THF (10 mL) was added dropwise (very slowly). After the addition, 4-fluoro-nitrobenezene (14.5 mL, 137 mmol) was added. The mixture was then stirred at room temperature overnight. EtOAc and 1N HCl were added. The organic layer was separated, dried over MgSO4, concentrated in vacuo. The residue was purified by a flash column using 50% EtOAc in hexane as eluents to give a solid (12.3 g).

A mixture of the solid (10.5 g, 54.1 mmol) in EtOH (75 mL) and conc. HCl (25 mL) was heated at reflux for 2 h. After cooling down, the precipitates were collected by filtration, washed with H₂O and EtOH, dried on vacuum (7.90 g).

To a solution of RaNi (50% slurry in H2O, 6 mL) and the solid (7.90 g, 44.6 mmol) in EtOH (150 mL), hydrazine hydrate (4.35 mL, 92.5 mmol) was added. N₂ gas evolved. After being stirred at room temperature overnight, the mixture was filtered through celite. The filtrate was concentrated in vacuo. The residue was purified by a flash column using 20 to 25% EtOAc in hexane as eluents to give the 2-methylbenzo[b]furan-5-ylamine (3.28 g). MS 148.1 (M+H).

A mixture of 2-methylbenzo[b]furan-5-ylamine (2.02 g, 13.7 mmol) and 1,1′-thiocarbonyldiimidazole (2.45 g, 13.8 mmol) in CH₂Cl₂ (20 mL) was stirred at room temperature for 3 h. After being concentrated in vacuo, the residue was applied to a short silica gel plug, which was eluted with hexane to give the desired product as a white solid (1.86 g).

### B. Preparation of N-[4-(dimethylaminoimino)phenyl]-2-[(2-methylbenzo[b]furan-5-yl)aminothiocarbonylamino]-acetamide

To a solution of N-Boc-glycine (1.75 g, 10.0 mmol) and 4-aminobenzonitrile (1.18 g, 10.0 mmol) in CH₂Cl₂ (10 mL), EDC (1.95 g, 10.2 mmol) was added. The mixture was stirred at room temperature overnight. The product was collected as a white precipitate by filtration (2.46 g). MS 276.1 (M+H) and 298.1 (M+Na).

To a solution of the nitrile compound (630 mg, 2.29 mmol) in pyridine (10 mL) and TEA (1.0 mL), H2S gas was bubbled until saturation was reached. The solution was then stirred at room temperature overnight. It was concentrated in vacuo. The residue was dissolved in acetone (20 mL). Iodomethane (1.40 mL, 22.6 mmol) was added. It was heated at reflux for 1 h, then concentrated in vacuo. The residue was dissolved in MeOH (10 mL). To one half of the solution (5 mL), dimethylamine (2N in THF, 5.7 mL, 11.5 mmol) was added. The mixture was stirred at room temperature overnight. After being concentrated in vacuo, the residue was dissolved in TFA (5 mL). It was then stirred at room temperature for 1 h. After being concentrated in vacuo, the residue was purified by HPLC to give white powder (172 mg). MS 221.1 (M+H).

A solution of the white powder (71 mg, 0.32 mmol), 2-methylbenzo[b]furan-5-yl isothiocyanate (prepared in Part A, 61 mg, 0.32 mmol) and TEA (0.18 mL, 1.3 mmol) in CH₂Cl₂ (6 mL) was stirred at room temperature 3 h. It was then concentrated in vacuo. The residue was purified by HPLC to give the titled compound as a powder (56 mg). MS 410.1 (M+H).

### Example 2

### N-[4-(1-methyl-4,5-dihydro-1H-imidazol-2-yl)phenyl]-2-[(2-methylbenzo[b)furan-5-yl)aminothiocarbonylamino]-acetamide

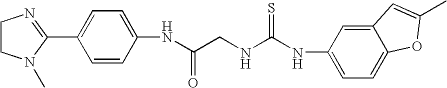

To another half of the thioimidate solution (from Part B of Example 1, 5 mL), N-methylethylenediamine (1.0 mL, 11.4 mmol) was added. The mixture was heated at reflux for 1 h, then was stirred at room temperature overnight. After being concentrated in vacuo, the residue was dissolved in TFA (5 mL). It was then stirred at room temperature for 1 h. After being concentrated in vacuo, the residue was purified by HPLC to give white powder (185 mg).

A solution of the white powder (21 mg, 0.091 mmol), 2-methylbenzo[b]furan-5-yl isothiocyanate (prepared in Part A, 17 mg, 0.090 mmol) and TEA (0.50 mL, 3.6 mmol) in CH₂Cl₂ (5 mL) was stirred at room temperature overnight. It was then concentrated in vacuo. The residue was purified by HPLC to give the titled compound as a powder (15 mg). MS 422.1 (M+H).

### Example 3

### N-[4-(1-methyl-4,5-dihydro-1H-imidazol-2-yl)phenyl]-2-isobutyl-2-[(2-methylbenzo[b]furan-5-yl)aminothiocarbonylamino]-acetamide

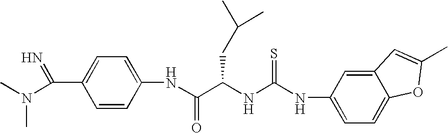

To a solution of N-Boc-L-leucine (647 mg, 2.60 mmol) and 4-aminobenzonitrile (307 mg, 2.60 mmol) in dry pyridine (10 mL) at room temperature, POCl₃ (0.484 mL, 5.19 mmol) was added. After being stirred for 3 h, EtOAc and H₂O were added. The organic layer was separated, washed with 1N HCl, dried over Na₂SO₄, concentrated in vacuo to give an oil (550 mg).

To a solution of the oil (540 mg, 1.63 mmol) in pyridine (8 mL) and TEA (0.8 mL), H2S gas was bubbled until saturation was reached. The solution was then stirred at room temperature overnight. It was concentrated in vacuo. The residue was dissolved in acetone (15 mL). Iodomethane (1.0 mL, 16.1 mmol) was added. It was heated at reflux for 30 min, then concentrated in vacuo. The residue was dissolved in MeOH (10 mL). To the solution, dimethylamine (2N in THF, 4.0 mL, 8.0 mmol) was added. The mixture was heated at reflux for 30 min. After being concentrated in vacuo, the residue was dissolved in TFA (10 mL). It was then stirred at room temperature for 1 h. After being concentrated in vacuo, the residue was purified by HPLC to give white powder (330 mg). MS 277.2 (M+H).

A solution of the white powder (52 mg, 0.13 mmol), 2-methylbenzo[b]furan-5-yl isothiocyanate (prepared in Part A of Example 1, 25 mg, 0.13 mmol) and TEA (0.056 mL, 0.40 mmol) in CHCl₃ (4 mL) and CH₃CN (2 mL) was stirred at room temperature overnight. It was then concentrated in vacuo. The residue was purified by HPLC to give the titled compound as a powder (25 mg). MS 466.2 (M+H).

### Example 4

### N-[4-(dimethylaminoimino)phenyl]-2-phenyl-2-(4-bromophenylaminothiocarbonylamino)-acetamide

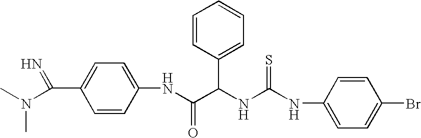

To a solution of N-Boc-phenylglycine (1.00 g, 4.00 mmol) and 4-aminobenzonitrile (0.470 g, 4.00 mmol) in dry pyridine (10 mL) at 0 C, POCl₃ (0.80 mL, 9.72 mmol) was added. After being stirred at 0 C for 30 min, EtOAc and H₂O were added. The organic layer was separated, washed with 1N HCl, sat. NaHCO3, brine, dried over Na₂SO₄, concentrated in vacuo to give an oil (1.40 g).

To a solution of the oil (1.38 g, 3.93 mmol) in dioxane (30 mL) and TEA (4 mL), H2S gas was bubbled until saturation was reached. The solution was then stirred at room temperature for three days. It was concentrated in vacuo. The residue was dissolved in acetone (30 mL). Iodomethane (2.5 mL, 40.1 mmol) was added. It was heated at reflux for 3 h, then concentrated in vacuo. The residue was dissolved in MeOH (30 mL). To one sixth of the thioimidate solution (5.0 mL), a pre-mixed solution of dimethylamine (2N in THF, 1.7 mL, 3.4 mmol) and HOAc (0.30 mL, 5.2 mmol) was added. The mixture was stirred at room temperature overnight. After being concentrated in vacuo, the residue was dissolved in TFA (7 mL). It was then stirred at room temperature for 1 h. After being concentrated in vacuo, the residue was purified by HPLC to give white powder (125 mg).

A solution of the white powder (33 mg, 0.11 mmol), 4-bromophenyl isothiocyanate (31 mg, 0.14 mmol) and TEA (0.040 mL, 0.29 mmol) in THF (3 mL) was stirred at room temperature overnight. It was then concentrated in vacuo. The residue was purified by HPLC to give the titled compound as a powder (8 mg). MS 510.1 and 512.1 (M+H, Br pattern).

### Example 5

### N-[4-(pyrrolidinylimino)phenyl]-2-phenyl-2-(4-bromophenylaminothiocarbonylamino)-acetamide

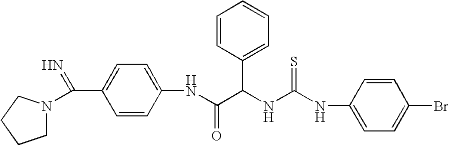

To the thioimidate solution (from Example 4, 5.0 mL, 0.66 mmol), a pre-mixed solution of pyrrolidine (0.273 mL, 3.3 mmol) and HOAc (0.28 mL, 4.9 mmol) was added. The mixture was stirred at room temperature overnight. After being concentrated in vacuo, the residue was dissolved in TFA (7 mL). It was then stirred at room temperature for 1 h. After being concentrated in vacuo, the residue was purified by HPLC to give a white powder (185 mg), as a TFA salt of N-[4-(pyrrolidinylimino)phenyl]-2-phenyl-2-amino-acetamide.

A solution of the white powder (36 mg, 0.11 mmol), 4-bromophenyl isothiocyanate (31 mg, 0.14 mmol) and TEA (0.040 mL, 0.29 mmol) in THF (3 mL) was stirred at room temperature overnight. It was then concentrated in vacuo. The residue was purified by HPLC to give the titled compound as a powder (13 mg). MS 536.1 and 538.1 (M+H, Br pattern).

### Example 6

### N-[4-(azetidinylimino)phenyl]-2-phenyl-2-(4-bromophenylaminothiocarbonylamino)-acetamide

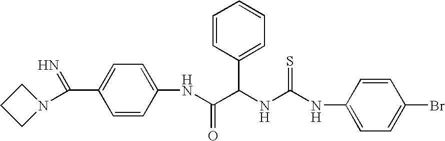

To the thioimidate solution (from Example 4, 5.0 mL, 0.66 mmol), a solution of azetidine HOAc (2M in MeOH, 1.7 mL, 3.4 mmol) was added. The mixture was stirred at room temperature overnight. After being concentrated in vacuo, the residue was dissolved in TFA (7 mL). It was then stirred at room temperature for 1 h. After being concentrated in vacuo, the residue was purified by HPLC to give white powder (180 mg).

A solution of the white powder (35 mg, 0.11 mmol), 4-bromophenyl isothiocyanate (31 mg, 0.14 mmol) and TEA (0.040 mL, 0.29 mmol) in THF (3 mL) was stirred at room temperature overnight. It was then concentrated in vacuo. The residue was purified by HPLC to give the titled compound as a powder (12 mg). MS 522.1 and 524.0 (M+H, Br pattern).

### Example 7

### N-{4-[(N-methyl-N-2-methoxyethyl)aminoimino]phenyl}-2-phenyl-2-(4-bromophenylaminothiocarbonylamino)-acetamide

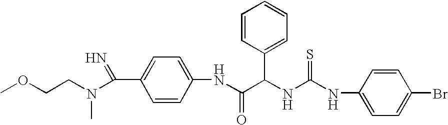

To the thioimidate solution (from Example 4, 5.0 mL, 0.66 mmol), a pre-mixed solution of N-(methoxyethyl)methylamine (0.35 mL, 3.3 mmol) and HOAc (0.28 mL, 4.9 mmol) was added. The mixture was stirred at room temperature overnight. After being concentrated in vacuo, the residue was dissolved in TFA (7 mL). It was then stirred at room temperature for 1 h. After being concentrated in vacuo, the residue was purified by HPLC to give white powder (112 mg).

A solution of the white powder (35 mg, 0.10 mmol), 4-bromophenyl isothiocyanate (30 mg, 0.14 mmol) and TEA (0.040 mL, 0.29 mmol) in THF (3 mL) was stirred at room temperature overnight. It was then concentrated in vacuo. The residue was purified by HPLC to give the titled compound as a powder (24 mg). MS 554.1 and 556.1 (M+H, Br pattern).

### Example 8

### N-[4-(4-ethoxycarbonylpiperidinylimino)phenyl]-2-phenyl-2-(4-bromophenylaminothiocarbonylamino)-acetamide

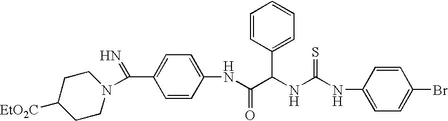

To the thioimidate solution (from Example 4, 5.0 mL, 0.66 mmol), a pre-mixed solution of ethyl isonipecolate (0.50 mL, 3.3 mmol) and HOAc (0.28 mL, 4.9 mmol) was added. The mixture was stirred at room temperature overnight. After being concentrated in vacuo, the residue was dissolved in TFA (7 mL). It was then stirred at room temperature for 1 h. After being concentrated in vacuo, the residue was purified by HPLC to give white powder (165 mg).

A solution of the white powder (41 mg, 0.10 mmol), 4-bromophenyl isothiocyanate (32 mg, 0.15 mmol) and TEA (0.040 mL, 0.29 mmol) in THF (3 mL) was stirred at room temperature overnight. It was then concentrated in vacuo. The residue was purified by HPLC to give the titled compound as a powder (5 mg). MS 622.1 and 624.1 (M+H, Br pattern).

### Example 9

### N-[4-(1-methyl-4,5-dihydro-1H-imidazol-2-yl)phenyl]-2-phenyl-2-(4-bromophenylaminothiocarbonylamino)-acetamide

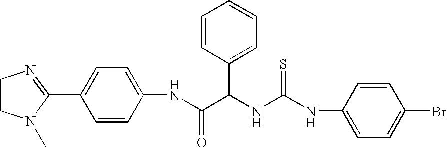

To the thioimidate solution (from Example 4, 5.0 mL, 0.66 mmol), a pre-mixed solution of N-methylethylenediamine (0.29 mL, 3.3 mmol) and HOAc (0.56 mL, 9.8 mmol) was added. The mixture was heated at reflux for 4 h. After being concentrated in vacuo, the residue was dissolved in TFA (7 mL). It was then stirred at room temperature for 1 h. After being concentrated in vacuo, the residue was purified by HPLC to give white powder (148 mg).

A solution of the white powder (38 mg, 0.12 mmol), 4-bromophenyl isothiocyanate (33 mg, 0.15 mmol) and TEA (0.040 mL, 0.29 mmol) in THF (3 mL) was stirred at room temperature overnight. It was then concentrated in vacuo. The residue was purified by HPLC to give the titled compound as a powder (27 mg). MS 522.1 and 524.1 (M+H, Br pattern).

### Example 10

### N-[4-(pyrrolidinylimino)phenyl]-2-phenyl-2-(4-fluorophenylaminothiocarbonylamino)-acetamide

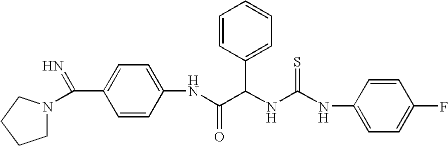

A solution of N-[4-(pyrrolidinylimino)phenyl]-2-phenyl-2-amino-acetamide (from Example 5, TFA salt, 36 mg, 0.11 mmol), 4-fluorophenyl isothiocyanate (27 mg, 0.18 mmol) and TEA (0.040 mL, 0.29 mmol) in THF (3 mL) was stirred at room temperature overnight. After being concentrated in vacuo, the residue was purified by HPLC to give the titled compound as a powder (6 mg). MS 476.2 (M+H).

### Example 11

### N-[4-(pyrrolidinylimino)phenyl]-2-phenyl-2-(4-ethynylphenylaminothiocarbonylamino)-acetamide

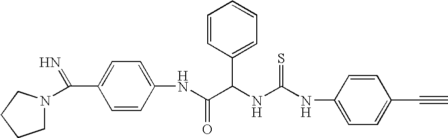

To a solution of thiocarbonyldiimidazole (0.500 g, 90%, 2.53 mmol) in CHCl₃ (5 mL), 4-ethynylaniline (0.305 g, 97%, 2.53 mmol) was added. After being stirred at room temperature overnight, the solution was concentrated in vacuo to give a solid (0.805 g).

A solution of the solid (50 mg, containing imidazole, 0.17 mmol), N-[4-(pyrrolidinylimino)phenyl]-2-phenyl-2-amino-acetamide (from Example 5, TFA salt, 36 mg, 0.11 mmol) and TEA (0.040 mL, 0.29 mmol) in THF (3 mL) was stirred at room temperature overnight. After being concentrated in vacuo, the residue was purified by HPLC to give the titled compound as a powder (8 mg). MS 482.2 (M+H).

### Example 12

### N-[4-(pyrrolidinylimino)phenyl]-2-phenyl-2-(5-bromopyridin-2-ylaminothiocarbonylamino)-acetamide

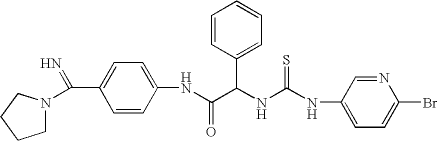

To a solution of thiocarbonyldiimidazole (1.12 g, 90%, 5.66 mmol) in CHCl₃ (8 mL), 2-amino-5-bromopyridine (1.00 g, 5.78 mmol) was added. After being stirred at room temperature overnight, the solution was concentrated in vacuo to give a brown solid (2.10 g), as 5-bromopyridin-2-yl isothiocyanate.

A solution of the solid (51 mg, containing imidazole, 0.14 mmol), N-[4-(pyrrolidinylimino)phenyl]-2-phenyl-2-amino-acetamide (from Example 5, TFA salt, 36 mg, 0.11 mmol) and TEA (0.040 mL, 0.29 mmol) in THF (3 mL) was stirred at room temperature overnight. After being concentrated in vacuo, the residue was purified by HPLC to give the titled compound as a powder (14 mg). MS 537.1 and 539.1 (M+H, Br pattern).

### Example 13

### (S) N-[4-(pyrrolidinylimino)phenyl]-2-phenyl-2-(4-bromophenylaminothiocarbonylamino)-acetamide

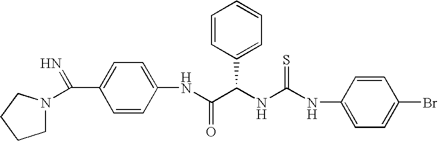

The titled compound was synthesized analogously as described in Example 4, using L isomer of N-Boc-phenylglycine in the place of the racemates, and using pyrrolidine instead of dimethylamine. MS 536.1 and 538.1 (M+H, Br pattern).

### Example 14

### (S) N-[4-(piperidinylimino)phenyl]-2-phenyl-2-(4-bromophenylaminothiocarbonylamino)-acetamide

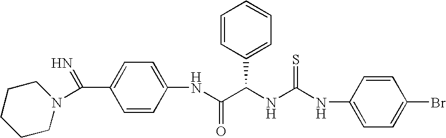

The titled compound was synthesized analogously as described in Example 4, using L isomer of N-Boc-phenylglycine in the place of the racemates, and using piperidine instead of dimethylamine. MS 550.1 and 552.1 (M+H, Br pattern).

### Example 15

### (S) N-[4-(homopiperidinylimino)phenyl]-2-phenyl-2-(4-bromophenylaminothiocarbonylamino)-acetamide

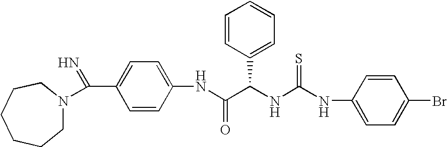

The titled compound was synthesized analogously as described in Example 4, using L isomer of N-Boc-phenylglycine in the place of the racemates, and using homopiperidine instead of dimethylamine. MS 564.1 and 566.2 (M+H, Br pattern).

### Example 16

### (S) N-[4-(pyrrolidinylimino)phenyl]-2-phenyl-2-(4-chlorophenylaminothiocarbonylamino)-acetamide

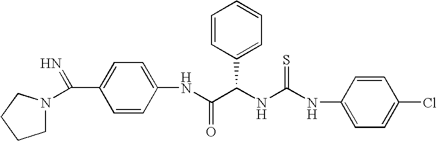

The titled compound was synthesized analogously as described in Example 4, using L isomer of N-Boc-phenylglycine in the place of the racemates, and using pyrrolidine instead of dimethylamine, and using 4-chlorophenyl isothiocyanate instead of 4-bromophenyl isothiocyanate. MS 492.1 and 494.1 (M+H, Cl pattern).

### Example 17

### (S) N-[4-(pyrrolidinylimino)phenyl]-2-phenyl-2-(2-fluoro-4-bromophenylaminothiocarbonylamino)-acetamide

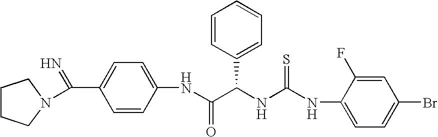

The titled compound was synthesized analogously as described in Example 4, using L isomer of N-Boc-phenylglycine in the place of the racemates, and using pyrrolidine instead of dimethylamine, and using 2-fluoro-4-bromophenyl isothiocyanate instead of 4-bromophenyl isothiocyanate. MS 554.1 and 556.1 (M+H, Br pattern).

### Example 18

### (S) N-[4-(pyrrolidinylimino)phenyl]-2-phenyl-2-(2-trifluoromethoxy-4-bromophenylaminothiocarbonylamino)-acetamide

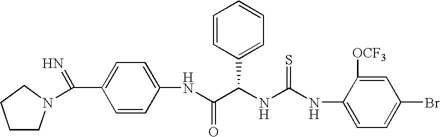

The titled compound was synthesized analogously as described in Example 4, using L isomer of N-Boc-phenylglycine in the place of the racemates, and using pyrrolidine instead of dimethylamine, and using 2-trifluoromethoxy-4-bromophenyl isothiocyanate instead of 4-bromophenyl isothiocyanate. MS 620.1 and 622.1 (M+H, Br pattern).

### Example 19

### (S) N-[4-(pyrrolidinylimino)phenyl]-2-phenyl-2-(2-methyl-4-bromophenylaminothiocarbonylamino)-acetamide

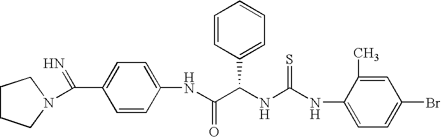

The titled compound was synthesized analogously as described in Example 4, using L isomer of N-Boc-phenylglycine in the place of the racemates, and using pyrrolidine instead of dimethylamine, and using 2-methyl-4-bromophenyl isothiocyanate instead of 4-bromophenyl isothiocyanate. MS 550.1 and 552.1 (M+H, Br pattern).

### Example 20

### (S) N-[4-(pyrrolidinylimino)phenyl]-2-phenyl-2-(2,4-dichlorophenylaminothiocarbonylamino)-acetamide

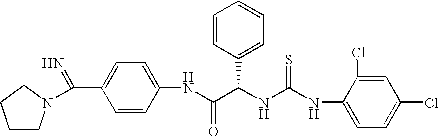

The titled compound was synthesized analogously as described in Example 4, using L isomer of N-Boc-phenylglycine in the place of the racemates, and using pyrrolidine instead of dimethylamine, and using 2,4-dichlorophenyl isothiocyanate instead of 4-bromophenyl isothiocyanate. MS 526.1, 527.2 and 528.2 (M+H, 2Cl pattern).

### Example 21

### (S) N-[4-(pyrrolidinylimino)phenyl]-2-phenyl-2-(3-chloro-4-bromophenylaminothiocarbonylamino)-acetamide

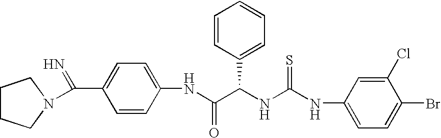

The titled compound was synthesized analogously as described in Example 4, using L isomer of N-Boc-phenylglycine in the place of the racemates, and using pyrrolidine instead of dimethylamine, and using 3-chloro-4-bromophenyl isothiocyanate instead of 4-bromophenyl isothiocyanate. MS 570.2, 571.1 and 572.0 (M+H, Cl+Br pattern).

### Example 22

### (S) N-[4-(pyrrolidinylimino)phenyl]-2-phenyl-2-(2-trifluoromethyl-4-bromophenylaminothiocarbonylamino)-acetamide

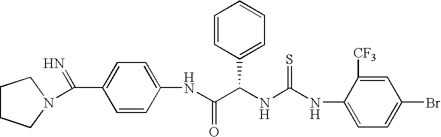

The titled compound was synthesized analogously as described in Example 4, using L isomer of N-Boc-phenylglycine in the place of the racemates, and using pyrrolidine instead of dimethylamine, and using 2-trifluoromethyl-4-bromophenyl isothiocyanate instead of 4-bromophenyl isothiocyanate. MS 604.1 and 606.1 (M+H, Br pattern).

### Example 23

### (S) N-[4-(pyrrolidinylimino)phenyl]-2-phenyl-2-(2,4,6-tribromophenylaminothiocarbonylamino)-acetamide

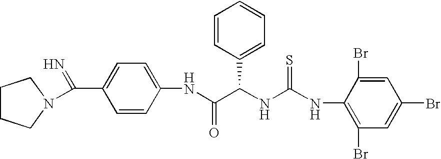

The titled compound was synthesized analogously as described in Example 4, using L isomer of N-Boc-phenylglycine in the place of the racemates, and using pyrrolidine instead of dimethylamine, and using 2,4,6-tribromophenyl isothiocyanate instead of 4-bromophenyl isothiocyanate. MS 693.9 (M+H, 3Br pattern).

### Example 24

### N-[4-(1-methyl-4,5-dihydro-1H-imidazol-2-yl)phenyl]-2-isopropyl-2-(4-chlorophenylaminothiocarbonylamino]-acetamide

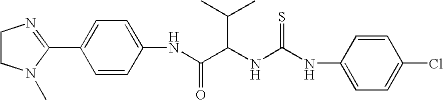

To a solution of N-Boc-valine (217 mg, 1.00 mmol) and 4-aminobenzonitrile (118 mg, 1.00 mmol) in dry pyridine (5 mL) at 0 C, POCl₃ (0.186 mL, 2.00 mmol) was added. After being stirred at room temperature overnight, EtOAc and H₂O were added. The organic layer was separated, washed with 1N HCl, dried over Na₂SO₄, concentrated in vacuo to give a brownish solid (260 mg).

To a solution of the solid (260 mg, 0.820 mmol) in MeOH (5 mL) at 0 C, hydrogen chloride gas was bubbled through until saturation was reached. After being stirred at room temperature overnight, the solution was concentrated in vacuo. The residue was dissolved in MeOH (6 mL). To the solution, N-methylethylenediamine (0.36 mL, 4.1 mmol) was added. After being heated at reflux for 2 h, the solution was concentrated in vacuo. The residue was purified by HPLC to give an oil (90 mg).

A solution of the oil (45 mg, 0.16 mmol), 4-chlorophenyl isothiocyanate (40 mg, 0.24 mmol) and TEA (0.050 mL, 0.36 mmol) in THF (4 mL) was stirred at room temperature overnight. It was concentrated in vacuo. The residue was purified by HPLC to give the titled compound as a powder (23 mg). MS 444.2 and 446.1 (M+H, Cl pattern).

### Example 25

### N-[4-(3-oxo-morpholin-4-yl)phenyl]-2-phenyl-2-(4-chlorophenylaminothiocarbonylamino)-acetamide

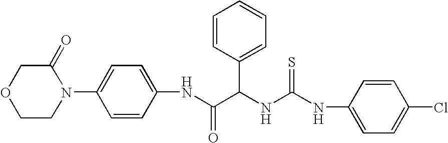

### A. Preparation of 4-(3-oxo-morpholin-4-yl)phenylamine

NaH (60%, 3.2 g, 80 mmol) in a flask was washed with hexane. To the flask cooled in an ice-bath, a solution of ethanolamine (4.4 mL, 73 mmol) in dioxane (40 mL) was added. The mixture was heated at reflux for 10 min until no H₂ gas evolved. The thick slurry was then cooled in an ice-bath, and a solution of ethyl chloroacetate (8.9 g, 73 mmol) in dioxane (15 mL) was added. The reaction mixture was heated at reflux for 1 h. It was then filtered. The filtrate was concentrated in vacuo to give an oil, which was purified by a short flash column, eluted with EtOAc/MeOH (95/5) to give a white solid (1.9 g), as 3-morpholinone.

To a blue solution of 3-morpholinone (250 mg, 2.48 mmol), 4-iodoaniline (650 mg, 2.97 mmol), CuI (47 mg, 0.25 mmol) and N,N′-dimethylethylenediamine (0.040 mL, 0.372 mmol) in dioxane (5 mL) in a pressure bottle, K₂CO₃ (683 mg, 4.95 mmol) was added. The mixture was heated at 110 C overnight. After being cooled to room temperature, the crude dark solution was loaded to two preparative TLC plates, eluted with EtOAc/MeOH (95/5) to give the desired product as off-white solid (240 mg). MS 193.1 (M+H).

### B. Preparation of N-[4-(3-oxo-morpholin-4-yl)phenyl]-2-phenyl-2-(4-chlorophenylaminothiocarbonylamino)-acetamide

To a solution of N-Boc-phenylglycine (52 mg, 0.21 mmol) and 4-(3-oxo-morpholin-4-yl)phenylamine (40 mg, 0.21 mmol) in pyridine (3 mL) at 0 C, POCl₃ (0.048 mL, 0.52 mmol) was added. After being stirred at 0 C for 2 h, H₂O and EtOAc were added. The organic layer was separated, dried over Na₂SO₄, concentrated in vacuo. The residue was purified by HPLC to the desired product as a white powder (14 mg).

A solution of the powder (14 mg, 0.033 mmol) in TFA (2 mL) was stirred at room temperature for 2 h. After being concentrated in vacuo, the residue was dissolved in DMF (1 mL). To the solution, 4-chlorophenyl isothiocyanate (12 mg, 0.070 mmol) and TEA (0.10 mL, 0.72 mmol) were added. After being stirred at room temperature overnight, the solution was concentrated in vacuo. The residue was purified by HPLC to give the titled compound as a white powder (8 mg). MS 495.2 and 497.2 (M+H, Cl pattern).

### Example 26

### (S) N-[4-(1-methylpiperidin-4-yl)piperazin-1-yl]-2-phenyl-2-(4-chlorophenylaminothiocarbonylamino)-acetamide

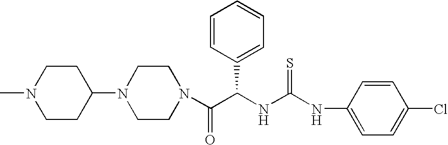

To a solution of N-Boc-L-phenylglycine (50 mg, 0.20 mmol), 1-(1-methylpiperidin-4-yl)piperazine (purchased from Oakwood products, 38 mg, 0.21 mmol) and TEA (0.055 mL, 0.40 mmol) in DMF (2 mL), BOP (116 mg, 0.26 mmol) was added. After being stirred at room temperature overnight, EtOAc and 5% aq. NaHCO₃ were added. The organic layer was separated, dried over Na₂SO₄, concentrated in vacuo. The residue was dissolved in TFA (4 mL). After being stirred at room temperature for 2 h, the solution was concentrated in vacuo. The residue was dissolved in DMF (2 mL). To the solution, 4-chlorophenyl isothiocyanate (51 mg, 0.30 mmol) and TEA (0.85 mL) were added. After being stirred at room temperature for 30 min, the solution was concentrated in vacuo. The residue was purified by HPLC to give the titled compound as a white powder (41 mg). MS 486.2 and 488.2 (M+H, Cl pattern).

### Example 27

### (R) N-[4-(1-methylpiperidin-4-yl)piperazin-1-yl]-2-phenyl-2-(4-chlorophenylaminothiocarbonylamino)-acetamide

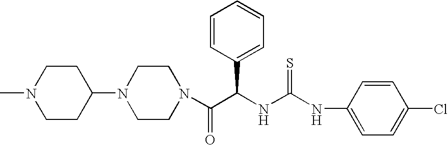

The titled compound was synthesized analogously as described in Example 26, using N-Boc-D-phenylglycine in the place of N-Boc-L-phenylglycine. MS 486.2 and 488.2 (M+H, Cl pattern).

### Example 28

### N-[4-(2-aminosulfonylphenyl)phenyl]-2-phenyl-2-[(2-methylbenzo[b]furan-5-yl)aminothiocarbonylamino]-acetamide

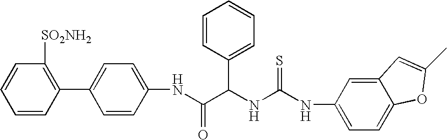

### A. Preparation of 4-[(2-tert-butylaminosulfonyl)phenyl]-aniline

To a solution of tert-butylamine (5.73 g, 78.4 mmol) and triethylamine (16.6 mL, 119 mmol) in CH₂Cl₂ (200 mL) in an ice bath, benzenesulfonyl chloride (13.85 g, 78.4 mmol) was added dropwise. The mixture was stirred at room temperature overnight. It was washed with saturated Na₂CO₃ (60 mL) and brine (60 mL). The organic layer was separated, and the aqueous layer was extracted with CH₂Cl₂ (2×50 mL). The combined organic extracts were dried over MgSO₄. The solvent was evaporated in vacuo to give the titled compound as a light yellowish solid (15.9 g). MS 214 (M+H).

To a solution of the solid (15.9 g, 74.7 mmol) in THF (200 mL) in an ice bath, n-butyllithium in hexane (1.6 M, 100 mL, 164 mmol) was added dropwise, followed by addition of triisopropylborate (24.1 mL, 104 mmol) dropwise. The mixture was stirred at room temperature for 4 h. While being cooled in an ice bath, 1N hydrochloride (200 mL) was added. The mixture was stirred at room temperature overnight. It was extracted with ether (2×50 mL). The organic extract was washed with 1N sodium hydroxide (2×60 mL). The aqueous solution was acidified to pH=1 with 6N hydrochloride, and then extracted with ether (2×100 ml). The ether extract was dried over MgSO4, and concentrated in vacuo to give the titled compound as a white solid (11.5 g). MS 258 (M+H).

A mixture of the white solid (6.00 g, 23.4 mmol), 4-bromoaniline (4.01 g, 23.3 mmol) and Pd(Ph₃P)₄ (1.35 g, 1.17 mmol) in toluene (120 mL), water (16 mL), isopropanol (60 mL), and aq. NaOH (SM, 40 mL) was heated to reflux for 6 h. After being cooled down, the reaction mixture is partitioned between water and ethyl acetate. The organic layer was separated, dried over MgSO₄, and concentrated in vacuo. The residue was purified by a silica gel flash column using 30% ethyl acetate in hexanes as eluents to give a solid (5.06 g). MS 305 (M+H).

### B. Preparation of N-[4-(2-aminosulfonylphenyl)phenyl]-2-phenyl-2-[(2-methylbenzo[b]furan-5-yl)aminothiocarbonylamino]-acetamide

To a solution of N-Boc-phenylglycine (128 mg, 0.510 mmol) and 4-[(2-tert-butylaminosulfonyl)phenyl]-aniline (311 mg, 1.02 mmol) in dry pyridine (5 mL) at room temperature, POCl₃ (0.20 mL, 2.43 mmol) was added. After being stirred at room temperature for 15 min, EtOAc and H₂O were added. The organic layer was separated, washed with 1N HCl, sat. NaHCO₃, brine, dried over Na₂SO₄, concentrated in vacuo to give solid (410 mg).

A solution of the solid (410 mg) in TFA (10 mL) was stirred at room temperature for 2 days. It was then concentrated in vacuo. The residue was dissolved in EtOAc. The solution was washed with sat. aq. NaHCO₃, brine, dried over Na₂SO₄, concentrated in vacuo to give a semi-solid (253 mg), as N-[4-(2-aminosulfonylphenyl)phenyl]-2-phenyl-2-amino-acetamide.

To a solution of the semi-solid (84 mg, 0.22 mmol) in CHCl₃ (4 mL) and CH₃CN (2 mL), 2-methylbenzo[b]furan-5-yl isothiocyanate (from Example 1, 42 mg, 0.22 mmol) was added. After being stirred at room temperature for 3 days, the mixture was concentrated in vacuo. The residue was purified by a silica column using 25 to 50% EtOAc in hexane as eluents to give the titled compound (15 mg). MS 571.1 (M+H).

### Example 29

### N-[4-(2-aminosulfonylphenyl)phenyl]-2-phenyl-2-[(4-bromophenyl)aminothiocarbonylamino]-acetamide

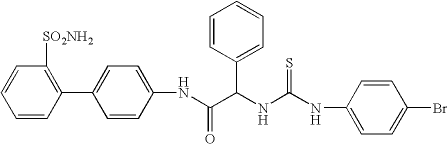

A solution of N-[4-(2-aminosulfonylphenyl)phenyl]-2-phenyl-2-amino-acetamide (from Example 28, 74 mg, 0.19 mmol), 4-bromophenyl isothiocyanate (47 mg, 0.22 mmol) and TEA (0.10 mL, 0.72 mmol) in CH₃CN (6 mL) and CH₂Cl₂ (2 mL) was stirred at room temperature overnight. After being concentrated in vacuo, the residue was purified by HPLC to give the titled compound (36 mg). MS 595.0 and 597.0 (M+H, Br pattern).

### Example 30

### (S) N-[4-(2-aminosulfonylphenyl)phenyl]-2-phenyl-2-(5-bromopyridin-2-ylaminothiocarbonylamino)-acetamide

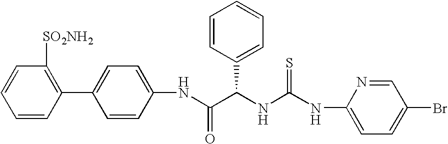

The titled compound was synthesized analogously as described in Example 28, using L-isomer of N-Boc-phenylglycine in the place of the racemates, and 5-bromopyridin-2-yl isothiocyanate (from Example 12) instead of 2-methylbenzo[b]furan-5-yl isothiocyanate. MS 618.0 and 620.0 (M+Na, Br pattern).

### Example 31

### (R) N-[4-(2-aminosulfonylphenyl)phenyl]-2-phenyl-2-(5-bromopyridin-2-ylaminothiocarbonylamino)-acetamide

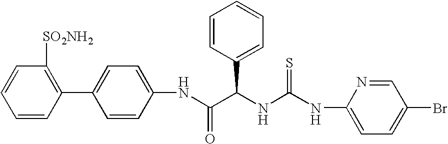

The titled compound was synthesized analogously as described in Example 28, using D-isomer of N-Boc-phenylglycine in the place of the racemates, and 5-bromopyridin-2-yl isothiocyanate (from Example 12) instead of 2-methylbenzo[b]furan-5-yl isothiocyanate. MS 618.0 and 620.0 (M+Na, Br pattern).

### Example 32

### (S) N-[4-(2-aminosulfonylphenyl)phenyl]-2-phenyl-2-(4-chlorophenylaminothiocarbonylamino)-acetamide

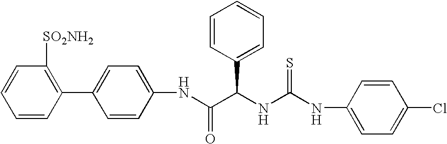

The titled compound was synthesized analogously as described in Example 28, using L-isomer of N-Boc-phenylglycine in the place of the racemates, and 4-chlorophenyl isothiocyanate instead of 2-methylbenzo[b]furan-5-yl isothiocyanate.

### Example 33

### (R) N-[4-(2-aminosulfonylphenyl)phenyl]-2-phenyl-2-(4-chlorophenylaminothiocarbonylamino)-acetamide

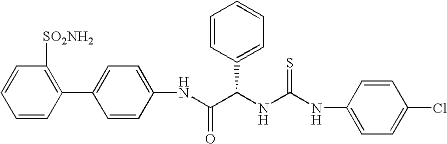

The titled compound was synthesized analogously as described in Example 28, using D-isomer of N-Boc-phenylglycine in the place of the racemates, and 4-chlorophenyl isothiocyanate instead of 2-methylbenzo[b]furan-5-yl isothiocyanate.

### Example 34

### Preparation of (2S) N-[4-(2-pyridon-1-yl)phenyl]-2-phenyl-2-(4-chlorophenylaminothiocarbonylamino)-acetamide

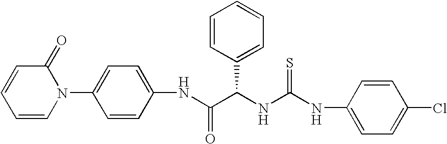

### A. Preparation of 4-(2-pyridon-1-yl)phenylamine

A mixture of 4-iodoaniline (1.00 g, 4.57 mmol), 2-hydroxypyridine (0.477 g, 5.02 mmol), 8-hydroxyquinoline (0.110 g, 0.759 mmol) and K₂CO₃ (0.945 g, 6.85 mmol) in DMSO (10 mL) was degassed with Ar before being charged with CuI (0.145 g, 0.763 mmol). The mixture in a sealed tube was then heated at 130° C. overnight. Water and nBuOH were added. The mixture was filtered. The nBuOH phase was separated, and concentrated in vacuo to give a solid (0.666 g), which was pure enough for subsequent reactions. MS 187.3 (M+H).

### B. Preparation of (2S) N-[4-(2-pyridon-1-yl)phenyl]-2-phenyl-2-(4-chlorophenylaminothiocarbonylamino)-acetamide

To a solution of (L) N-BOC-phenylglycine (126 mg, 0.500 mmol) and 4-(2-pyridon-1-yl)phenylamine (102 mg, 0.548 mmol) in DMF (3 mL), EDC (144 mg, 0.750 mmol) was added. The mixture was stirred at room temperature for 1 h. Water (10 mL) was added to induce precipitation. The precipitate was collected by filtration to give the amide (61 mg). MS 420.2 (M+H).

The amide (61 mg, 0.15 mmol) was dissolved in TFA (4 mL). After being stirred at room temperature for 1 h, TFA was removed in vacuo. The residue was partitioned between EtOAc and aq. 5% NaHCO₃. The EtOAc phase was separated, dried over Na₂SO₄, concentrated in vacuo to give a solid (35 mg).

To a solution of the solid (17 mg, 0.053 mmol) in CH₃CN (2 mL), 4-chlorophenylisothiocyanate (22 mg, 0.13 mmol) was added. After being stirred at room temperature for 30 min, the mixture was purified by HPLC to give the titled compound (6 mg). MS 489.0 and 491.0 (M+H, Cl pattern).

### Example 35

### Preparation of (2R) N-[4-(2-pyridon-1-yl)phenyl]-2-phenyl-2-(4-chlorophenylaminothiocarbonylamino)-acetamide

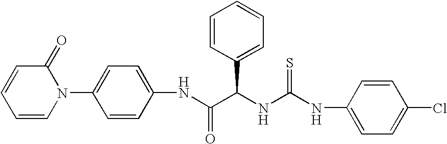

The titled compound was prepared analogously to the procedure described in Example 34, using (D) N-BOC-phenylglycine in the place of (L) N-BOC-phenylglycine. MS 489.2 and 491.2 (M+H, Cl pattern).

### Example 36

### Preparation of (2R) N-[4-(2-pyridon-1-yl)-2-fluorophenyl]-2-phenyl-2-(4-chlorophenylaminothiocarbonylamino)-acetamide

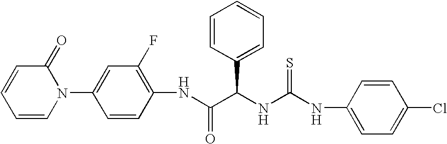

### A. Preparation of 4-(2-pyridon-1-yl)-2-fluorophenylamine

A mixture of 2-fluoro-4-iodoaniline (1.08 g, 4.56 mmol), 2-hydroxypyridine (0.477 g, 5.02 mmol), 8-hydroxyquinoline (0.110 g, 0.759 mmol) and K₂CO₃ (0.945 g, 6.85 mmol) in DMSO (10 mL) was degassed with Ar before being charged with CuI (0.145 g, 0.763 mmol). The mixture in a sealed tube was then heated at 130° C. overnight. Water and nBuOH were added. The mixture was filtered. The nBuOH phase was separated, and concentrated in vacuo to give a solid (0.902 g), which was pure enough for subsequent reactions. MS 205.2 (M+H).

### B. Preparation of (2R) N-[4-(2-pyridon-1-yl)-2-fluorophenyl]-2-phenyl-2-(4-chlorophenylaminothiocarbonylamino)-acetamide

The titled compound was prepared analogously to the procedure described in Example 35, using 4-(2-pyridon-1-yl)-2-fluorophenylamine in the place of 4-(2-pyridon-1-yl)phenylamine. MS 504.8 and 506.8 (M-H, Cl pattern).

### Example 37

### Preparation of (2R) 4-(1-methylpiperidin-4-yl)piperidin-1-yl-2-phenyl-2-(4-chlorophenylaminothiocarbonylamino)-acetamide

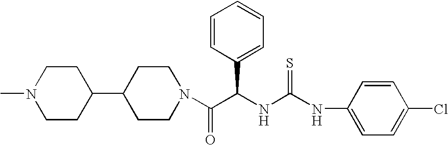

To a solution of (D) N-BOC phenylglycine (100 mg, 0.40 mmol), 4,4′-bipiperidine dihydrochloride (482 mg, 2.00 mmol) and triethylamine (0.70 mL, 5.0 mmol) in DMF (6.0 mL) and H₂O (3.0 mL), BOP (355 mg, 0.80 mmol) was added. After being stirred at room temperature for 1 h, the mixture was purified by HPLC to give the amide (160 mg). MS 402.5 (M+H).

To a solution of the amide (160 mg, 0.40 mmol) and HCHO (37% aq, 0.178 mL, 2.39 mmol) in MeOH (6.0 mL), NaBH₃CN (151 mg, 2.39 mmol) was added. After being stirred at room temperature overnight, the mixture was concentrated in vacuo. The residue was dissolved in TFA (6.0 mL). After being stirred for 2 h, TFA removed in vacuo. The residue was dissolved in CH₃CN (6 mL). To the solution, 4-chlorophenylisothiocyanate (110 mg, 0.64 mmol) was added. After 30 min of stirring, the mixture was purified by HPLC to give the titled compound (16 mg). MS 485.2 and 487.3 (M+H, Cl pattern).

### Example 38

This example illustrates methods for evaluating the compounds of the invention, along with results obtained for such assays. The in vitro and in vivo Factor Xa isoform activities of the inventive compounds can be determined by various procedures known in the art, such as a test for their ability to inhibit the activity of the Factor Xa isoform. The potent affinities for Factor Xa isoform exhibited by the inventive compounds can be measured by an IC₅₀ value (in nM). The IC₅₀ value is the concentration (in nM) of the compound required to provide 50% inhibition of Factor Xa isoform. The smaller the IC₅₀ value, the more active (potent) is a compound for inhibiting Factor Xa isoform.

An in vitro assay for detecting and measuring inhibition activity against Factor Xa is as follows:

IC₅₀ and Ki Determinations:

Substrate:

The substrate S-2765 (Z-D-Arg-Gly-Arg-pNA.HCl) was obtained from Diapharma (West Chester, Ohio).

Enzyme:

The human plasma protein factor Xa was purchased from Haematologic Technologies (Essex Junction, Vt.).

Methods

IC₅₀ Determinations

All assays, which are performed in 96-well microtiter plates, measure proteolytic activity of the enzyme (factor Xa) by following cleavage of paranitroanilide substrate. The assay buffer used for proteolytic assays was Tris buffered saline (20 mM Tris, 150 mM NaCl, 5 mM CaCl₂, 0.1% Bovine serum albumin (BSA), 5% Dimethly Sulfoxide (DMSO) pH 7.4). In a 96-well microtiter plate, inhibitor was serially diluted to give a range of concentrations from 0.01 nM to 10 μM (final). Duplicate sets of wells were assayed and control wells without inhibitor were included. Enzyme was added to each well, (fXa concentration=1 nM), the plate was shaken for 5 seconds and then incubated for 5 minutes at room temperature. S2765 was added (100 μM final) and the plate was shaken for 5 seconds (final liquid volume in each well was 200 μl). The degree of substrate hydrolysis was measured at 405 nm on a Thermomax plate reader (Molecular Devices, Sunnyvale, Calif.) for 2 minutes. The initial velocities (mOD/min), for each range of inhibitor concentrations, were fitted to a four parameter equation using Softmax data analysis software. The parameter C, derived from the resulting curve-fit, corresponded to the concentration for half maximal inhibition (IC₅₀).

Kᵢ Determination

The assay buffer for this series of assays was Hepes buffered saline (20 mM Hepes, 150 mM NaCl, 5 mM CaCl₂, 0.1% PEG-8000, pH 7.4). In a 96-well microtiter plate, inhibitor was serially diluted in a duplicate set of wells to give a range of final concentrations from 5 pM to 3 μM final. Controls without inhibitor (8 wells) were included. The enzyme, fXa (1 nM final) was added to the wells. The substrate S-2765 (200 μM final) was added and the degree of substrate hydrolysis was measured at 405 nm on a Thermomax plate reader for 5 minutes, using Softmax software. Initial velocities (mOD/min) were analyzed by non-linear least squares regression in the Plate Ki software (BioKin Ltd, Pullman, Wash.) (Literature reference: Kusmic P, Sideris S, Cregar L M, Elrod K C, Rice K D, Janc J. High-throughput screening of enzyme inhibitors: Automatic determination of tight-binding inhibition constants. Anal. Biochemistry 2000, 281:62-67). The model used for fitting the inhibitor dose-response curves was the Morrison equation. An apparent Kᵢ (Ki*) was determined. The overall Kᵢ was calculated using the following equation:

$$Ki = \frac{{Ki}^{*}}{1 + \frac{[ S ]}{Km}}$$

where [S] is substrate concentration (200 μM) and Kₘ, the Michaelis constant for S2765.

The hERG (Human Ether-A-Go-Go Related Gene Protein) Membrane Binding Assay

Human embryonic kidney (HEK293) cells stably transfected with hERG cDNA were used for preparation of membranes (Literature reference: Zhou, Z., Gong, Q., Ye, B., Fan, Z., Makielski, C., Robertson, G., January, C T., Properties of hERG stably expressed in HEK293 cells studied at physiological temperature. Biophys. J, 1998, 74:230-241). The assay buffer was comprised of 50 mM Tris, 10 mM KCl, 1 mM MgCl₂, pH 7.4. Competition assays for hERG binding were performed, in a 96 well plate, with 50 μL ³H— dofetilide, at a concentration of 3.5 nM (final concentration of 0.01% ethanol). Test compound was added at final concentrations of 100 μM, 33.33 μM, 11.11 μM, 3.70 μM, 1.23 μM, 0.41 μM, 0.14 μM, 0.046 μM, 0.015 μM, and 0.005 μM (1.0% DMSO). Each compound was run in duplicate on each of two plates. Total binding was determined by addition of 50 μL of assay buffer in place of compound. Non-specific binding was determined by addition of 50 mL of 50 μM terfenadine in place of test compound. All assays were initiated by addition of 150 μL of membrane homogenates (15 μg protein/well as final concentration) to the wells (total volume=250 μL per well), and the plates were incubated at room temperature for 80 minutes on a shaking platform. All assays were terminated by vacuum filtration on to glass fiber filters, followed by two washes with cold assay buffer. The filter plates were dried at 55° C. for 90 minutes, after which, Microscint 0 (50 μL) was added to each well of the dried filter plate. The plates were counted on a Packard Topcount (Perkin Elmer, Boston, Mass.) using a one minute protocol. Scintillation reading (counts per minute, CPM) data generated by the Packard TopCount was used to calculate the percent inhibition of ³H-dofetilide binding, for each compound at each concentration, using the total binding control value corrected for non-specific binding. The IC₅₀ value was calculated from the percent inhibition curve generated using Excel XL Fit software (Microsoft). The equilibrium dissociation constant (Kᵢ) was calculated using the equation of Cheng and Prusoff (see “Relationship between the inhibition constant (Kᵢ) and the concentration of inhibitor which causes 50 percent inhibition (I₅₀) of an enzymatic reaction,” Biochem Pharmacol., 1973, 22(23):3099-108.

ti Kᵢ=IC₅₀/[1+([L]/K_{D}).

A compound can be run through this assay and its corresponding IC₅₀ inhibition value can be calculated from the assay data.

The following examples exhibited Factor Xa IC₅₀ values less than or equal to 100 nM: 4, 5, 6, 7, 9, 10, 11, 12, 13, 15, 16, 24, 25, 26, 27, 28, 29 30, 33, 34, 35 and 36.

The following examples exhibited Factor Xa IC₅₀ values greater than 100 nM and less than 500 nM: 14, 31 and 32.

The following examples exhibited Factor Xa IC₅₀ values greater than or equal to 500 nM: 1, 2, 3, 8, 17, 18, 19, 20, 21, 22, 23 and 37.

The present invention provides a number of embodiments. It is apparent that the examples may be altered to provide other embodiments of this invention. Therefore, it will be appreciated that the scope of this invention is to be defined by the appended claims rather than by the specific embodiments, which have been represented by way of example.

1. A compound having the formula: 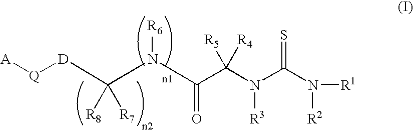
wherein:
R¹ is a member selected from the group consisting of: hydrogen, —C₁₋₆alkyl, —C₀₋₆alkyl-aryl, heteroaryl and —C₂₋₆alkenyl;
R² is a member selected from the group consisting of: —C₀₋₆alkyl-aryl, —C₃₋₈cycloalkylaryl, heteroaryl, heteroaryl-C₃₋₈cycloalkyl, —C₃₋₈cycloalkyl, —C₃₋₈cycloalkenyl, heteromonocyclyl, fused heterobicyclyl and unfused heterobicyclyl, each of which is optionally substituted with from 1 to 3 R^{2a} substituents, wherein each heteromonocyclyl, fused heterobicyclyl or unfused heterobicyclyl comprises 5 to 12 ring atoms, 1 to 4 of which are members independently selected from the group consisting of N, O and S;
R³ is a member selected from the group consisting of: hydrogen, C₁₋₆alkyl, heteroaryl, C₂₋₆alkenyl, —C₀₋₄alkyl-C₃₋₈-cycloalkyl, —C₀₋₆alkyl-aryl, —C₀₋₆alkyl-heteroaryl, —C₀₋₆alkyl-heterocyclyl, —C₀₋₆alkyl-CO—OR^{3a}, —C₁₋₆alkyl-N(R^{3a}R^{3b}), —C₁₋₆alkyl-O—R^{3a}, —C₁₋₆alkyl-S—R^{3a}, —C₀₋₆alkyl-C(O)—N(R^{3a}R^{3b}) and —C₁₋₆alkyl-N(R^{3a})—C(O)R^{3b};
each R⁴ and R⁵ is a member independently selected from the group consisting of: hydrogen, —C₁₋₆alkyl, —C₂₋₆alkenyl, —C₂₋₆alkynyl, —C₀₋₄alkyl-C₃₋₈-cycloalkyl, C₁₋₆haloalkyl, —C₀₋₆alkyl-heteroaryl, —C₀₋₆alkyl-heterocyclyl, —C₀₋₆alkyl-CN, —C₀₋₆alkyl-NO₂, —C₁₋₆alkyl-O—R^{4a}, —C₁₋₆alkyl-S—R^{4a}, —C₁₋₆alkyl-SO₂—R^{4a}, —C₁₋₆alkyl-S(O)—R^{4a}, —C₀₋₆alkyl-CO—OR^{4a}—C₀₋₆alkyl-C(O)—N(R^{4a}R^{4b}), —C₀₋₆alkyl-C(O)R^{4a}, —C₁₋₆alkyl-N(R^{4a}R^{4b}), —C₁₋₆alkyl-N(R^{4a})—C(O)R^{4b}, —C₁₋₆alkyl-N(R^{4a})—C(O)—N(R^{4b}R^{4c}), —C₁₋₆alkyl-N(R^{4a})—SO₂—R^{4b}, —C₁₋₆alkyl-SO₂—N(R^{4a}R^{4b}), —C₀₋₆alkyl-PO(—OR^{4a})(—OR^{4b}), —C₁₋₆alkyl-N(R^{4a})—PO(—OR^{4b})(—OR^{4c}), —C₀₋₆alkyl-aryl, —C₀₋₆alkyl-heteroaryl, and —C₀₋₆alkyl-heterocyclyl; or R⁴ and R⁵ can be taken together with the carbon atom to which they are attached to form a 3 to 8 membered heterocyclyl group; wherein each heterocyclyl is a 3 to 8 membered monocyclic ring or a 8-12 membered bicyclic ring, each comprising from 1 to 4 heteroatoms selected from the group consisting of O, N and S, and wherein 1 to 3 carbon or nitrogen atoms of aryl, heteroaryl and heterocyclyl are substituted with 1 to 3 R^{4d} substituents;
D is a member selected from the group consisting of: a direct bond, aryl, heteroaryl, C₃₋₈cycloalkyl, C₃₋₈cycloalkylene, heteromonocyclyl, unfused heterobicyclyl, and fused heterobicyclyl; each of which is optionally substituted with 1 to 3 R⁹ substituents, wherein each heteromonocyclyl, fused heterobicyclyl or unfused heterobicyclyl comprises from 5 to 10 ring atoms, 1-4 of which are selected from the group consisting of N, O and S;
Q is selected from the group consisting of: a direct bond, —C(R^{10a}R^{10b}), —C(O)—, —C(S)—, —C(═NR^{10a})—, —O—, —S—, —N(R^{10a})—, —N(R^{10a})CH₂—, —CH₂N(R^{10a})—, —C(O)N(R^{10a})—, —N(R^{10a})C(O)—, —SO₂—, —SO—, —SO₂N(R^{10a})—, and —N(R^{10a})—SO₂—; and at least one of D and Q is not a direct bond;
A is selected from the group consisting of: —NR^{11c}R^{11d}, —C(═NR^{11c})NR^{11a}R^{11b}, —C(═NR^{11e}R^{11f})NR^{11a}R^{11b}, —N(R^{11d})C(═NR^{11c})NR^{11a}R^{11b}, —N(R^{11d})C(═NR^{11c})R^{11a}, —N(R^{11c})NR^{11a}R^{11b}, —N(R^{11c})OR^{11d}; C₁₋₆alkyl, C₂₋₆alkenyl, aryl, heteroaryl, —C₃₋₈cycloalkyl, —C₃₋₈cycloalkenyl, heteromonocyclyl, and fused heterobicyclyl; each of aryl, heteroaryl, heteromonocyclyl and fused heterobicyclyl, each of which is optionally substituted with 1 to 3 R^{11g}; wherein each hetercyclyl comprises from 5 to 10 ring atoms, 1-4 of which are selected from the group consisting of N, O and S;
each R^{2a}, R^{4d}, R⁹ and R^{11g} is a member independently selected from the group consisting of: H, halo, C₁₋₆alkyl, C₂₋₆alkenyl, C₂₋₆alkynyl, C₀₋₄alkylC₃₋₉cycloalkyl, —C₁₋₄alkoxy, —O—C₀₋₂alkyl-CF₃, —C₀₋₂alkyl-CF₃, —C₀₋₂alkyl-CN, —C₀₋₂alkyl-NO₂, —C₀₋₂alkyl-NR^{12a}R^{12b}, —C₀₋₂alkyl-SO₂NR^{12a}R^{12b}, —C₀₋₂alkyl-SO₂R^{12a}, —C₀₋₂alkyl-SOR^{12a}, —C₀₋₂alkyl-CF₃, —C₀₋₂alkyl-OR^{12a}, —C₀₋₂alkyl-SR^{12a}, —O—CH₂—CH₂—OR^{12a}, —O—CH₂—CO₂R^{12a}, —N(R^{12a})—CH₂—CH₂—OR^{12b}, —C₀₋₂alkyl-C(O)NR^{12a}R^{12b}, —C₀₋₂alkyl-CO₂R^{12a}, —C₀₋₂alkyl-N(R^{12a})—C(O)R^{12b}, —C₀₋₂alkyl-N(R^{12c})—C(O)NR^{12a}R^{12b}, —C₀₋₂alkyl-C(═NR^{12c})NR^{12a}R^{12b}, —C₀₋₂alkyl-C(═NR^{12a})R^{12b}, —C₀₋₂alkyl-N(R^{12d})C(═NR^{12c})NR^{12a}R^{12b}, —C₀₋₂alkyl-N(R^{12a})—SO₂—R^{12b}, ═O, ═S, ═NR^{12a}, 5- or 6-membered aryl, 5- or 6-membered heteroaryl and 5- to 7-membered heterocyclyl, each of which is optionally substituted with a member independently selected from the group consisting of halo, CF₃, OCF₃, SCF₃, C₁₋₆alkyl, C₂₋₆alkenyl, C₂₋₆alkynyl, C₀₋₄alkylC₃₋₈cycloalkyl, C₁₋₄alkoxy, —CO₂H, —CO₂C₁₋₄alkyl, —CONR^{12a}R^{12b}, ═O, ═S, —OH, —CN and —NO₂; wherein each heteroaryl or heterocyclyl comprises 1 to 4 heteroatoms, independently selected from the group consisting of N, O and S,
each R^{3a}, R^{3b}, R^{4a}, R^{4b}, R^{4c}, R^{11a}, R^{11b}, R^{11c}, R^{11d}, R^{11e}, R^{11f}, R^{12a}, R^{12b}, R^{12c} and R^{12d} are members independently selected from the group consisting of: H, C₁₋₆alkyl, C₂₋₆alkenyl, C₂₋₆alkynyl, C₀₋₄alkylC₃₋₈cycloalkyl, C₀₋₄alkylaryl, C₀₋₄alkyl-heteroaryl, —C₀₋₆alkyl-COC₁₋₄alkyl, —C₀₋₆alkyl-CO₂C₁₋₄alkyl, —C₀₋₆alkyl-SO₂—C₁₋₄alkyl, —C₀₋₆alkyl-SO₂—N(C₁₋₄alkyl, C₁₋₄alkyl), —C₀₋₆alkyl-N(C₁₋₄alkyl, C₁₋₄alkyl) and —C₁₋₆alkyl-O—C₀₋₆alkyl, wherein 1-3 hydrogen atoms on the aryl or heteroaryl ring may be independently replaced with a member selected from the group consisting of halo, C₁₋₆alkyl, C₂₋₆alkenyl, C₂₋₆alkynyl, C₀₋₄alkylC₃₋₈cycloalkyl, C₁₋₄alkoxy, —CO₂H, —CO₂C₁₋₄alkyl, —CON(C₁₋₄alkyl, C₁₋₄alkyl), —OH, —CN and NO₂; or can be taken together with the nitrogen atom to which they are attached to form a 3-8 membered heterocyclyl group, comprising 1 to 4 heteroatoms selected from the group consisting of N, O and S, each of which is optionally substituted with 1 to 4 R¹³ substituents selected from the group consisting of halo, C₁₋₆alkyl, C₂₋₆alkenyl, C₂₋₆alkynyl, C₀₋₄alkylC₃₋₈cycloalkyl, C₀₋₄alkoxy, —CO₂H, —CO₂C₁₋₄alkyl, —CON(C₁₋₄alkyl, C₁₋₄alkyl), ═O, ═S, —OH, —CN and NO₂;
each R⁶, R⁷, R⁸, R^{10a} and R^{10b} is a member independently selected from the group consisting of: hydrogen, C₁₋₆alkyl, C₂₋₆alkenyl, C₂₋₆alkynyl and C₀₋₄alkylC₃₋₈cycloalkyl, —C₀₋₆alkyl-aryl and —C₀₋₆alkyl-heteroaryl; or R⁴ and R⁶ can be taken together with the atoms to which they are attached to form a 5 to 12 membered heterocyclyl group; wherein each heterocyclyl is a 5 to 8 membered monocyclic ring or a 8-12 membered bicyclic ring, each comprising from 1 to 4 heteroatoms selected from the group consisting of O, N and S, and wherein 1 to 3 carbon or nitrogen atoms of aryl, heteroaryl and heterocyclyl are substituted with 1 to 3 R^{4d} substituents;
each subscript n1 and n2 is an integer of 0 to 1; and
pharmaceutically acceptable salts, solvates, hydrates, and prodrugs thereof.

2. A compound of claim 1, wherein R¹ and R³ is H.

3. A compound of claim 1, wherein R² is aryl or heteroaryl, each of which is optionally substituted with 1 to 3 R^{2a}.

4. A compound of claim 3, wherein R² is selected from the group consisting of phenyl, pyridyl and benzofuranyl.

5. A compound of claim 4, wherein each optional substituent R^{2a} is independently selected from the group consisting of halo, C₁₋₆alkyl, C₂₋₆alkynyl, —C₁₋₄alkoxy, —O—C₀₋₂alkyl-CF₃ and —C₀₋₂alkyl-CF₃.

6. A compound of claim 5, wherein R^{2a} is attached to the phenyl or pyridyl ring at a position para to the rest of the molecule.

7. A compound of claim 1, wherein R⁴ and R⁵ is a member independently selected from the group consisting of: hydrogen, —C₁₋₆alkyl and —C₀₋₆alkyl-aryl.

8. A compound of claim 7, wherein R⁴ is hydrogen and R⁵ is a member independently selected from the group consisting of hydrogen, isopropyl, isobutyl and phenyl.

9. A compound of claim 1, wherein when R⁴ and R⁵ are different the carbon bearing R⁵ has the R-configuration.

10. A compound of claim 1, wherein when R⁴ and R⁵ are different the carbon bearing R⁵ has the S-configuration.

11. A compound of claim 1, wherein n1 is 0.

12. A compound of claim 1, wherein n1 is 1.

13. A compound of claim 1, wherein R⁶ is H or R⁴ and R⁶ can be taken together with the atoms to which they are attached to form a 5 to 12 membered heterocyclyl group; wherein each heterocyclyl is a 5 to 8 membered monocyclic ring or a 8-12 membered bicyclic ring, each comprising from 1 to 4 heteroatoms selected from the group consisting of O, N and S, and wherein 1 to 3 carbon or nitrogen atoms of aryl, heteroaryl and heterocyclyl are substituted with 1 to 3 R^{4d} substituents.

14. A compound of claim 1, wherein R⁴ and R⁶ are taken together with the atoms to which they are attached selected from the group having the formula: 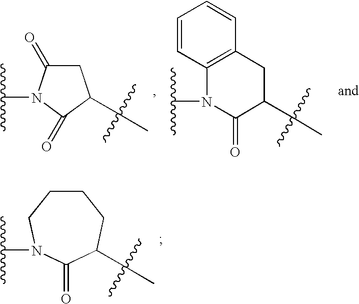
each of which is optionally substituted with 1 to 3 R^{4d} substituents.

15. A compound of claim 1, wherein n2 is 0.

16. A compound of claim 1, wherein n2 is 1.

17. A compound of claim 1, wherein each R⁷ and R⁸ is H.

18. A compound of claim 1, wherein D is aryl or heteromonocyclyl, wherein each heterocyclyl comprises from 5 to 7 ring atoms, 1 to 2 of which are N or O.

19. A compound of claim 18, wherein D is piperidinyl.

20. A compound of claim 18, wherein D is phenyl or piperazinyl.

21. A compound of claim 1, wherein Q is a direct bond or —C(═NH)—.

22. A compound of claim 20, wherein Q is attached to the phenyl or piperazinyl ring at a position para to the rest of the molecule.

23. A compound of claim 1, wherein A is selected from the group consisting of: —NR^{11a}R^{11b}, aryl, heteroaryl and heteromonocyclyl; each of aryl, heteroaryl, heteromonocyclyl and fused heterobicyclyl, each of which is optionally substituted with 1 to 3 R^{11g}; wherein each hetercyclyl comprises from 5 to 7 ring atoms, 1 to 2 of which are selected from the group consisting of N and O.

24. A compound of claim 23, wherein A is pyridinyl.

25. A compound of claim 23, wherein A is a member selected from the group consisting of dihydroimidazolyl, pyrrolidinyl, azetidinyl, piperidinyl, homopiperidinyl, morpholinyl and phenyl.

26. A compound of claim 25, wherein each optional substituent R^{11g} is independently selected from the group consisting of halo, C₁₋₆alkyl, C₂₋₆alkynyl, —O—C₀₋₂alkyl-CF₃, —C₀₋₂alkyl-CF₃ and ═O.

27. A compound of claim 1, wherein A-Q-D-(CR⁷R⁸)ₙ₂—NR⁶ₙ₁ is selected from the group consisting of: 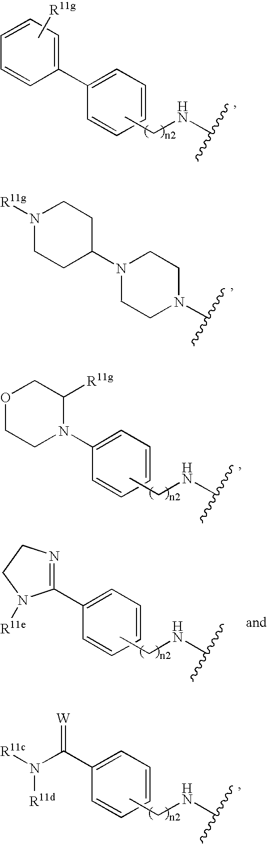
wherein W is O, S or NH; and the wavy line indicates the point of attachment to the rest of the molecule.

28. A compound of claim 1, wherein A-Q-D-(CR⁷R⁸)ₙ₂—NR⁶ₙ₁ is selected from the group consisting of: 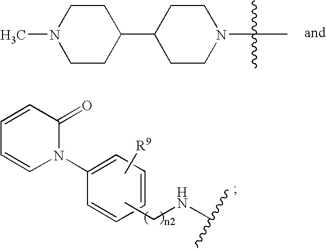
wherein the wavy line indicates the point of attachment to the rest of the molecule.

29. A compound of claim 1, wherein A-Q- is selected from the group consisting of: 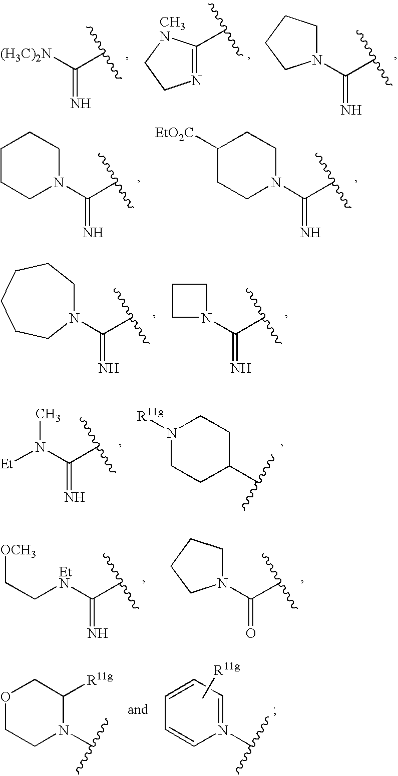
wherein the wavy line indicates the point of attachment to the rest of the molecule.

30. A compound of claim 26, wherein A-Q- is selected from the group consisting of: 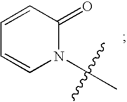
wherein the wavy line indicates the point of attachment to the rest of the molecule.

31-66. (canceled)

67. A compound selected from the group consisting of: N-[4-(dimethylaminoimino)phenyl]-2-[(2-methylbenzo[b]furan-5-yl)aminothiocarbonylamino]-acetamide; N-[4-(1-methyl-4,5-dihydro-1H-imidazol-2-yl)phenyl]-2-[(2-methylbenzo[b]furan-5-yl)aminothiocarbonylamino]-acetamide; N-[4-(1-methyl-4,5-dihydro-1H-imidazol-2-yl)phenyl]-2-isobutyl-2-[(2-methylbenzo[b]furan-5-yl)aminothiocarbonylamino]-acetamide; N-[4-(dimethylaminoimino)phenyl]-2-phenyl-2-(4-bromophenylaminothiocarbonylamino)-acetamide; N-[4-(pyrrolidinylimino)phenyl]-2-phenyl-2-(4-bromophenylaminothiocarbonylamino)-acetamide; N-[4-(azetidinylimino)phenyl]-2-phenyl-2-(4-bromophenylaminothiocarbonylamino)-acetamide; N-{4-[(N-methyl-N-2-methoxyethyl)aminoimino]phenyl}-2-phenyl-2-(4-bromophenylaminothiocarbonylamino)-acetamide; N-[4-(4-ethoxycarbonylpiperidinylimino)phenyl]-2-phenyl-2-(4-bromophenylaminothiocarbonylamino)-acetamide; N-[4-(1-methyl-4,5-dihydro-1H-imidazol-2-yl)phenyl]-2-phenyl-2-(4-bromophenylaminothiocarbonylamino)-acetamide; N-[4-(pyrrolidinylimino)phenyl]-2-phenyl-2-(4-fluorophenylaminothiocarbonylamino)-acetamide; N-[4-(pyrrolidinylimino)phenyl]-2-phenyl-2-(4-ethynylphenylaminothiocarbonylamino)-acetamide; N-[4-(pyrrolidinylimino)phenyl]-2-phenyl-2-(S-bromopyridin-2-ylaminothiocarbonylamino)-acetamide; (S) N-[4-(pyrrolidinylimino)phenyl]-2-phenyl-2-(4-bromophenylaminothiocarbonylamino)-acetamide; (S) N-[4-(piperidinylimino)phenyl]-2-phenyl-2-(4-bromophenylaminothiocarbonylamino)-acetamide; (S) N-[4-(homopiperidinylimino)phenyl]-2-phenyl-2-(4-bromophenylaminothiocarbonylamino)-acetamide; (S) N-[4-(pyrrolidinylimino)phenyl]-2-phenyl-2-(4-chlorophenylaminothiocarbonylamino)-acetamide; (S) N-[4-(pyrrolidinylimino)phenyl]-2-phenyl-2-(2-fluoro-4-bromophenylaminothiocarbonylamino)-acetamide; (S) N-[4-(pyrrolidinylimino)phenyl]-2-phenyl-2-(2-trifluoromethoxy-4-bromophenylaminothiocarbonylamino)-acetamide; (S) N-[4-(pyrrolidinylimino)phenyl]-2-phenyl-2-(2-methyl-4-bromophenylaminothiocarbonylamino)-acetamide; (S) N-[4-(pyrrolidinylimino)phenyl]-2-phenyl-2-(2,4-dichlorophenylaminothiocarbonylamino)-acetamide; (S) N-[4-(pyrrolidinylimino)phenyl]-2-phenyl-2-(3-chloro-4-bromophenylaminothiocarbonylamino)-acetamide; (S) N-[4-(pyrrolidinylimino)phenyl]-2-phenyl-2-(2-trifluoromethyl-4-bromophenylaminothiocarbonylamino)-acetamide; (S) N-[4-(pyrrolidinylimino)phenyl]-2-phenyl-2-(2,4,6-tribromophenylaminothiocarbonylamino)-acetamide; N-[4-(1-methyl-4,5-dihydro-1H-imidazol-2-yl)phenyl]-2-isopropyl-2-(4-chlorophenylaminothiocarbonylamino]-acetamide; N-[4-(3-oxo-morpholin-4-yl)phenyl]-2-phenyl-2-(4-chlorophenylaminothiocarbonylamino)-acetamide; (S) N-[4-(1-methylpiperidin-4-yl)piperazin-1-yl]-2-phenyl-2-(4-chlorophenylaminothiocarbonylamino)-acetamide; (R) N-[4-(1-methylpiperidin-4-yl)piperazin-1-yl]-2-phenyl-2-(4-chlorophenylaminothiocarbonylamino)-acetamide; N-[4-(2-aminosulfonylphenyl)phenyl]-2-phenyl-2-[(2-methylbenzo[b]furan-5-yl)aminothiocarbonylamino]-acetamide; N-[4-(2-aminosulfonylphenyl)phenyl]-2-phenyl-2-[(4-bromophenyl)aminothiocarbonylamino]-acetamide; (S) N-[4-(2-aminosulfonylphenyl)phenyl]-2-phenyl-2-(5-bromopyridin-2-ylaminothiocarbonylamino)-acetamide; (R) N-[4-(2-aminosulfonylphenyl)phenyl]-2-phenyl-2-(5-bromopyridin-2-ylaminothiocarbonylamino)-acetamide; (S) N-[4-(2-aminosulfonylphenyl)phenyl]-2-phenyl-2-(4-chlorophenylaminothiocarbonylamino)-acetamide; and (R) N-[4-(2-aminosulfonylphenyl)phenyl]-2-phenyl-2-(4-chlorophenylaminothiocarbonylamino)-acetamide; (2S) N-[4-(2-pyridon-1-yl)phenyl]-2-phenyl-2-(4-chlorophenylaminothiocarbonylamino)-acetamide; (2R) N-[4-(2-pyridon-1-yl)phenyl]-2-phenyl-2-(4-chlorophenylaminothiocarbonylamino)-acetamide; (2R) N-[4-(2-pyridon-1-yl)-2-fluorophenyl]-2-phenyl-2-(4-chlorophenylaminothiocarbonylamino)-acetamide; (2R) N-[4-(2-pyridon-1-yl)-2-fluorophenyl]-2-phenyl-2-(4-chlorophenylaminothiocarbonylamino)-acetamide; and (2R)4-(1-methylpiperidin-4-yl)piperidin-1-yl-2-phenyl-2-(4-chlorophenylaminothiocarbonylamino)-acetamide.

68. A composition comprising a pharmaceutically acceptable excipient and a compound of claim 1.

69. A method for preventing or treating a condition in a mammal characterized by undesired thrombosis comprising the step of administering to said mammal a therapeutically effective amount of a compound of claim 1.

70. A method in accordance with claim 69, wherein the condition is selected from the group consisting of acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, and conditions requiring the fitting of prosthetic devices.

71. A method for inhibiting the coagulation of a blood sample comprising contacting said sample with a compound of claim 1.
